(19) United States
(12) Patent Application Publication
(10) Pub. No.: US 2012/0116244 A1
(43) Pub. Date: May 10, 2012

(54) POSTURAL STABILITY IN PATIENTS HAVING A NEURO-DEGENERATIVE DISEASE USING A COMPUTATIONAL MODELING APPROACH TO DEEP BRAIN STIMULATION PROGRAMMING

(75) Inventors: Cameron C. McINTYRE, Cleveland, OH (US); Jay L. ALBERTS, Chagrin Halls, OH (US)

(73) Assignee: THE CLEVELAND CLINIC FOUNDATION, Cleveland, OH (US)

(21) Appl. No.: 13/288,684

(22) Filed: Nov. 3, 2011

Related U.S. Application Data
(63) Continuation-in-part of application No. 12/986,735, filed on Jan. 7, 2011.
Application No. 12/986,735 is a continuation-in-part of application No. PCT/US10/58770, filed on Dec. 2, 2010.
(60) Provisional application No. 61/265,782, filed on Dec. 2, 2009.
(60) Provisional application No. 61/409,693, filed on Nov. 3, 2010.

(51) Int. Cl. A61B 5/103; A61B 5/05

(52) U.S. Cl. 600/554; 600/587

(57) ABSTRACT

A system and method can provide for conducting a stimulation of anatomic regions to treat a neuromotor, neurocognitive or neuromotor and neurocognitive disorder, according to which stimulation, motor regions are stimulated, while creep of current to non-motor regions is minimized. Stimulation parameters can be selected based on at least one dual task test that involves cognitive and motor functions, wherein the motor function is postural stability.

### CROSS-REFERENCE TO RELATED APPLICATION

The present application is a continuation-in-part of U.S. patent application Ser. No. 12/986,735, filed Jan. 7, 2011, which is continuation-in-part of International Patent Application No. PCT/US10/58770, filed Dec. 2, 2010, which claims priority to U.S. Provisional Patent Application No. 61/265,782, filed Dec. 2, 2009, the entire contents of each of which is hereby incorporated by reference herein. The present application also claims priority to U.S. Provisional Patent Application No. 61/409,693, filed Nov. 3, 2010, the entire contents of which is hereby incorporated by reference herein.

### GOVERNMENT RIGHTS

Using the specific language required by 37 C.F.R. §401.14(0(4): This invention was made with government support under grant numbers R01 NS058706 and R01 NS059736 awarded by the National Science Foundation. The government has certain rights in the invention.

### FIELD OF THE INVENTION

The present invention relates to systems and methods for stimulating anatomic regions and/or for selecting parameters for such stimulation.

### BACKGROUND

A challenge faced by the majority of Parkinson's Disease (PD) patients is that, as the disease progresses, pharmacological therapy may no longer adequately treat postural instability and gait disturbances. Gait and balance disorders become a major limiting factor in the patients' quality of life and a potential source of severe injury from frequent falls. Falls are the main cause of disability and dependence, as approximately 70% of individuals with PD fall annually and 13% fall more than once a week (Wood, B. H. et al., “Incidence and prediction of falls in Parkinson's disease: a prospective multidisciplinary study,” J. Neurol. Neurosurg. Psychiatry 72(6), 721-5 (2002), the entire contents of which is hereby incorporated by reference herein). L-dopa therapy can improve gait and balance, but only early in the course of the disease.

Deep brain stimulation (DBS) in the subthalamic nucleus (STN) and other forms of neuromodulation are effective and safe surgical procedures that have been shown to reduce the motor dysfunction of advanced PD patients. Bilateral DBS refers to stimulation on both sides of the brain, while unilateral DBS refers to stimulation on one side of the brain. Bilateral and unilateral DBS typically target one of three areas, including the STN, globus pallidus interna (GPi), and ventral intermediate (Vim) nucleus of the thalamus. Bilateral STN DBS has been associated with declines in cognitive and cognitive-motor functioning. DBS is similarly used to treat other neuro-degenerative diseases including cognitive, motor, and cognitive-motor disorders, but presently used stimulation parameters result in detrimental side effects.

### SUMMARY

The present invention relates to the use of deep brain stimulation (DBS) of the subthalamic nucleus (STN) to assess postural stability under dual-task (working memory+postural stability) conditions using DBS parameters determined through Clinical DBS and Model DBS. Such assessment compares these two methods of DBS programming and evaluates cognitive-postural performance in an advanced PD patient. As described below, standardized clinical and biomechanical data were collected from one advanced PD patient under three stimulation conditions: Off, Clinical, and Model DBS. Model-based settings were selected to minimize the spread of current to non-motor regions of the STN, thereby focusing stimulation on areas previously shown to produce ideal therapeutic benefit.

According to an example embodiment of the present invention, a computer-implemented method includes selecting, by a computer processor, stimulation parameters for stimulating an anatomical region of a patient, the selection being based on results of at least one dual task test on which basis postural stability is assessed.

According to an example embodiment of the present invention, a computer-implemented method includes assessing, by a computer processor, a previously performed stimulation of an anatomical region of a patient by analyzing results of at least one dual task test, the results including an indication of postural stability.

According to an example embodiment of the present invention, a computer-readable medium has stored thereon instructions executable by a processor, the instructions which, when executed by the processor, cause the processor to perform a method, the method including selecting stimulation parameters for stimulating an anatomical region of a patient, the selection being based on results of at least one dual task test on which basis postural stability is assessed.

According to an example embodiment of the present invention, a system includes a computer processor configured to select stimulation parameters for stimulating an anatomical region of a patient, the selection being based on results of at least one dual task test on which basis postural stability is assessed.

### BRIEF DESCRIPTION OF THE DRAWINGS

FIG. 1 shows a patient specific model of deep brain stimulation (DBS), where a stereotactic coordinate system was defined relative to the imaging data, microelectrode recording data were entered into the model (thalamic cells, subthalamic cells, and substantia nigra cells, all represented by different ones of the dots), a three dimensional brain atlas was fitted to the neuroanatomy and neurophysiology (including representations of the thalamus and subthalamic nucleus), and a DBS electrode was positioned in the model, pertaining to a theoretical ellipsoid target volume, and referring to data presented for patient No 1.

FIG. 2 shows a representative volume of tissue activated (VTA) for the Right and Left stimulators during Clinical and Model DBS settings referring to data presented for patient No. 1, where A) Right side Model settings: contact 2, 2.0V, 0.06 ms, 130 Hz, B) Left side Model settings: contact 3, 1.8 V, 0.06 ms, 130 Hz, and C) Right side Clinical settings: contact 2-3+, 4.0V, 0.06 ms, 130 Hz. D) Left side Clinical settings: contact 2, 3.2V, 0.06 ms, 130 Hz.

FIG. 3 illustrates working memory performance as percent of letters correctly repeated during single- and dual-task conditions, pertaining to (A) results of the n-back task in the single-task condition at Off DBS, Clinical DBS and Model DBS (Means and Standard Errors), and (B) results of the n-back task in the dual-task condition at Off DBS, Clinical DBS and Model DBS (Means and Standard Errors), and where a cross marks a significant differences between Off and Clinical DBS, an asterisk marks a significant difference between Off and Model DBS, and a double asterisk marks a significant difference between Clinical and Model DBS.

FIG. 4 shows representative force-tracking trials (pertaining to patient 1) during Single (left-most column) and all Dual-task conditions (right columns) under the three DBS settings: Off (upper plots), Clinical DBS (middle plots) and Model DBS (lower plots), where the horizontal line represents the target force line the patient was instructed to match.

FIG. 5 shows force-tracking performance across stimulation conditions, where (A) results of the time within the target range (TWR) of force in the Single and Dual-task conditions at Off DBS, Clinical DBS and Model DBS (Means and Standard Errors), and (B) results of the relative root mean square error (RRMSE) force in the single and dual-task conditions at Off DBS, Clinical DBS and Model DBS (mod DBS) (Means and Standard Errors), and where a cross marks a significant differences between Off and Clinical DBS, an asterisk marks a significant difference between Off and Model DBS, and a double asterisk marks a significant difference between Clinical and Model DBS.

FIG. 6 shows dual-task losses (DTLs) and standard errors for (a) the force maintenance task (TWR), and (b) RRMSE at Off DBS, Clinical DBS (cli DBS), and Model DBS (mod DBS), where an asterisk signifies DTLs significantly greater then zero and significant differences between the states of stimulation (*p<0.05).

FIG. 7 is a flowchart showing a stimulation parameter selection method, according to an example embodiment of the present invention.

FIG. 8 illustrates working memory performance as percent of letters correctly repeated during dual-task conditions, pertaining to results of the n-back task in the dual-task condition at Off DBS, Clinical DBS and Model DBS according to a second study of the present invention.

FIG. 9 shows a patient specific model of deep brain stimulation (DBS) for the right side of the brain. Part A of FIG. 9 shows a 3D brain atlas fitted to the neuroanatomy (as defined by MRI) and neurophysiology (as defined by intraoperative electrophysiology) of the patient, and a representation of an estimated volume of tissue activated with clinical DBS stimulation settings. Part B of FIG. 9 shows a 3D brain atlas fitted to the neuroanatomy and neurophysiology of the patient, and a representation of an estimated volume of tissue activated with Model DBS stimulation settings and an implanted DBS electrode location and volume of tissue activated (red blob). Parts C-E of FIG. 9 show movement of the total body center of mass (red line) and center of pressure excursion (blue oval) for conditions of Off DBS, Clinical DBS and Model DBS, respectively.

### DETAILED DESCRIPTION

Activities of daily living are typically performed under modestly complex conditions and have cognitive and motor components that are performed simultaneously (Cahn-Weiner, D. A. et al., “Tests of executive function predict instrumental activities of daily living in community-dwelling older individuals,” Appl. Neuropsychol. 9, 187-91 (2002) (hereinafter “Cahn-Weiner et al., 2002,” the entire contents of which is hereby incorporated by reference herein). Frontal and executive dysfunction in the elderly and PD patients without DBS can be predictive of cognitive and motor function during Activities of Dailing Living (ADLs) (Cahn-Weiner et al., 2002; Cahn, D. A. et al., “Differential contributions of cognitive and motor component processes to physical and instrumental activities of daily living in Parkinson's disease,” Arch Clin Neuropsychol. 13, 575-83 (1998) (hereinafter “Cahn et al., 1998”), the entire contents of each of which is hereby incorporated by reference herein). Understanding how PD and DBS impact cognitive and motor function under conditions requiring greater cognitive rigor and during the simultaneous performance of cognitive and motor tasks can provide a more accurate assessment of the effect of a set of stimulation parameters on cognitive and motor performance when patients are completing “real world” tasks. Current methods of assessing cognitive and motor function in a clinical environment may not be sufficiently demanding or sensitive enough to reveal changes in cognitive or motor performance that occur when either component of a task is increased. There is an emerging body of literature indicating a paradox between the clinical improvements in motor functioning associated with STN DBS and the patient and caregiver's level of postoperative satisfaction (Agid, Y. et al., “Neurosurgery in Parkinson's disease: the doctor is happy, the patient less so?,” J. Neural Transm. Suppl. 409-14 (2006); Schupbach, M. et al., “Neurosurgery in Parkinson disease: a distressed mind in a repaired body?,” Neurology 66, 1811-6 (2006) (hereinafter “Schupbach et al., 2006”); Schupbach, M. et al., “Psychosocial adjustment after deep brain stimulation in Parkinson's disease,” Nature Clinical Practice 4, 58-59 (2008) (hereinafter “Schupbach and Agid, 2008”), the entire contents of each of which is hereby incorporated by reference herein).

Spread of current to non-motor areas of the STN can cause declines in cognitive and cognitive-motor functioning. As described in more detail below, a study was performed to assess and compare the cognitive-motor performance in advanced PD patients with bilateral STN DBS parameter settings determined clinically (Clinical), e.g., by subjective assessment such as asking a patient how the patient feels or observation of side effects due to stimulation, and with bilateral STN DBS parameter settings derived from a patient-specific computational model (Model), according to which current creep to non-motor regions was minimized or removed altogether. In this regard, the conventional method of parameter selection did not contemplate consideration of reduction of current creep to non-motor regions, but rather were selected largely on the subjective clinical measures, such that if one or more symptoms improved to some degree and there was no side effect noticed, then the parameters associated with those results were deemed worthy of use, without consideration of effect on creep to the non-motor regions. It was also conventionally not known that the spread of current to non-motor regions would negatively affect motor skill.

In the study, data were collected from 10 advanced PD patients, off medication, under three DBS conditions: OFF, Clinical and Model based stimulation. Clinical stimulation parameters had been determined based on clinical evaluations and the parameters were stable, i.e., unchanged, for at least six months prior to study participation. Model based parameters were selected to minimize the spread of current to non-motor portions of the STN using Cicerone DBS software (See Miocinovic S. et al., “Cicerone: stereotactice neurophysiological recording and deep brain stimulation electrode placement software system,” Acta Neurochir Suppl., 97, 561-7 (2007) (hereinafter “Miocinovic et al., 2007”), which is incorporated by reference herein). That is, in the study, and according to an example embodiment of the invention, software is used that provides a 2D or 3D visualization of patient images, DBS electrodes, and/or calculated estimated/predicted volumes of activation for specified stimulation parameters. Based on those visualizations, an operator is able to modify the parameters until a Volume of Tissue Activation (VTA) is provided that has minimal current spread to non-motor portions of the brain. In fact such visualization and modifications until parameters are selected for use can be performed without the presence of the patient.

For each stimulation condition (OFF, Clinical, and Model), participants performed a working memory (n-back task) and motor task (force-tracking) under single- and dual-task settings. During the dual-task, participants performed the n-back and force-tracking tasks simultaneously. Clinical and Model parameters were equally effective in improving the Unified Parkinson's disease Rating Scale (UPDRS-III) scores relative to Off DBS scores, e.g., with respect to motor response as measured by the UPDRS-III. The average improvement in off medication UPDRS-III scores for both parameter settings, 46 percent, is within the range of improvement typically reported in long-term studies with bilateral STN DBS in advanced PD patients (Abelson, J. L. et al., “Deep brain stimulation for refractory obsessive-compulsive disorder,” Biol Psychiatry. 57, 510-6 (2005); Kumar, R. et al., “Long-term follow-up of thalamic deep brain stimulation for essential and parkinsonian tremor,” Neurology. 61, 1601-4 (2003); Rodriguez-Oroz, M. C. et al., “Bilateral deep brain stimulation in Parkinson's disease: a multicentre study with 4 years follow-up,” Brain 128, 2240-9 (2005); Weaver, F. M. et al., “Bilateral deep brain stimulation vs best medical therapy for patients with advanced Parkinson disease: a randomized controlled trial,” Jama. 301, 63-73 (2009), the entire contents of each of which is hereby incorporated by reference herein). It was discovered that the n-back and force-tracking tasks, administered as described herein to obtain the test results described herein, provide a better context in which activities of daily living are completed, as most ADLs have a cognitive and motor component, and therefore provide a better measure than UPDRS for determining effectiveness of stimulation parameters.

Single-task working memory declines, in the 2-back condition, were significantly less under Model compared to Clinical DBS settings. Under dual-task conditions, force tracking was significantly better with Model compared to Clinical DBS. These results indicate that the cognitive and cognitive-motor declines associated with bilateral STN DBS can be reversed, without compromising motor benefits, by utilizing stimulation parameters that minimize current spread into non-motor regions of the STN. Theoretically, there can be a task that is 100% motor related, without any cognitive function required, in which case it can occur that there would be no performance difference between Clinical and Model parameters, but almost no ADLs are purely motor.

Indeed, the transmission of pathological information within the basal ganglia thalamocortical circuits is thought to underlie the symptoms of PD (Albin, R. L. et al., “The functional anatomy of basal ganglia disorders,” Trends Neurosci 12, 366-375 (1989); DeLong, M. R., “Primate models of movement disorders of basal ganglia origin,” Trends. Neurosci. 13, 281-285 (1990); Llinas, R. R. et al., “Thalamocortical dysrhythmia: a neurological and neuropsychiatric syndrome characterized by magnetoencephalography,” Proc. Natl. Acad. Sci. U.S.A. 96 (1999); Timmermann, L. et al., “The cerebral oscillatory network of parkinsonian resting tremor,” Brain 126, 199-212 (2003); Vitek, J. L. et al., “Physiology of hypokinetic and hyperkinetic movement disorders: model for dyskinesia,” Ann. Neurol. 47, S131-S140 (2000), the entire contents of each of which is hereby incorporated by reference herein). It is believed that DBS acts to regularize activity within the motor circuit thereby reducing the passage of pathological information from the pallidum (Grill, W. M. et al., “Deep brain stimulation creates an informational lesion of the stimulated nucleus,” Neuroreport 15, 1137-40 (2004); Guo, Y. et al., “Thalamocortical relay fidelity varies across subthalamic nucleus deep brain stimulation protocols in a data-driven computational model,” J. Neurophysiol. 99, 1477-92 (2008); Hashimoto, T. et al., “Stimulation of the subthalamic nucleus changes the firing pattern of pallidal neurons,” J. Neurosci. 23, 1916-23 (2003), the entire contents of each of which is hereby incorporated by reference herein). The spread of current to non-motor regions of the STN is likely to disrupt the spread of non-pathological information from these non-motor regions of the STN. Disruption of information processing in these non-motor regions could be responsible for the DBS related cognitive-motor declines observed under dual-task conditions. The loss of transmitted information or information processing capabilities may not produce a deficit in cognitive function following unilateral procedures (Alberts, J. L. et al., “Bilateral subthalamic stimulation impairs cognitive-motor performance in Parkinson's disease patients,” Brain 131, 3348-60 (2008) (herein after “Alberts et al., 2008”), the entire contents of which is hereby incorporated by reference) or when the patients are able to focus all of their attention on the performance of a cognitive or motor task alone, as is the case during most clinical examinations. However, as the cognitive demands of the task increase, information processing demands increase. Therefore, under bilateral STN DBS with conventional Clinically determined stimulation parameters, which results in spread of current to non-motor regions, such current spread can compromise cognitive-motor functioning. Cognitive resources on which patients can attempt to draw can now be even more compromised as a result of bilateral disruption of non-motor pathways. The Model parameters according to the present invention, set to avoid spread to non-motor regions, can minimize or avoid such further degeneration of cognitive resources.

Further in this regard, the focus of clinical programming has been on the motor response, and unless non-motor side effects are readily apparent, they are generally not detected; particularly those that can only arise under more complex testing conditions. In turn, unintentional over-stimulation can occur when the stimulus amplitude at a therapeutic contact is adjusted to be just below threshold for motor side effects, related to the assumption that more stimulation is better than less. However, the study conducted as described herein indicates that Model parameters resulted in similar improvements in clinical ratings and minimized cognitive-motor declines under dual-task conditions compared to Clinical settings, while using significantly less power (cf Table 4 below). Model parameters were selected to both focus a volume of tissue activated (VTA) on the target region and to be energy efficient. Previous clinical studies have found no significant benefit from using stimulation frequencies greater than 100-130 Hz (Moro, E. et al., “The impact on Parkinson's disease of electrical parameter settings in STN stimulation,” Neurology 59, 706-13 (2002) (hereinafter “Moro et al., 2002”; Rizzone, M. et al., “Deep brain stimulation of the subthalamic nucleus in Parkinson's disease: effects of variation in stimulation parameters,” J. Neurol. Neurosurg. Psychiatry 71, 215-9 (2001), the entire contents of each of which is hereby incorporated by reference herein), and from the biophysical perspective of axonal activation the most energy efficient pulse width available in the Medtronic Soletra/Kinetra DBS system is 60 μs in a monopolar configuration (Butson and McIntyre, 2007; Sahin, M. et al., “Non-rectangular waveforms for neural stimulation with practical electrodes,” J. Neural Eng. 4, 227-33 (2007), the entire contents of each of which is hereby incorporated by reference herein). Therefore, the Model DBS parameters according to the present invention can be selected with these constraints, resulting in reduced power consumption that could help to minimize the threat of stimulation induced tissue damage and prolong battery life expectancy.

In addition to better overall cognitive-motor performance associated with Model parameters, the amount of power consumed was, on average, less than half of the Clinical settings.

Although the present invention is described in relation to Parkinson's Disease and the STN, the methods and systems of the present invention can be used by patients suffering from other medical disorders and other anatomical sites. In preferred embodiments, the medical disorders are characterized by abnormal motor function, such as in patient's limbs (upper and/or lower extremities). The medical disorder can be a neurological disorder (i.e., a disorder of the patient's nervous system). In certain embodiments, the neurological disorder is a neuromotor or neurocognitive disorder that results in abnormal motor function and that is characterized by irregular motor cortical output including, for example, output from the cerebellum and/or supplementary motor area (“SMA”) of the cortex; and irregular sub-cortical output from regions that contribute to motor function in a patient such as, for example, the basal ganglia, the subthalamic nucleus and/or the thalamus.

The methods have application to mammalian patients, including humans suffering from the above-described disorders. In certain embodiments, the neuromotor or neurocognitive disorders are degenerative in nature. Exemplary disorders include PD, Alzheimer's Disorder, dementia, Parkinsonian syndrome, essential tremor, multiple sclerosis (MS), amyotrophic lateral sclerosis (ALS), traumatic brain injury, stroke, multiple system atrophy (MSA), and dystonia.

Embodiments of the present invention are directed to systems and methods for stimulating an anatomical region in a stimulation procedure in which less than 10% of non-motor regions (i.e., regions not associated with motor function), e.g., of the brain, are stimulated. In an example embodiment, one or more of the zona incerta, lenticular fasciculus, and motor region of the globus pallidus and internal capsule are stimulated, while less than 10% of non-motor anatomical-neural regions of the globus pallidus or STN are stimulated.

In an example embodiment of the present invention, an anatomical region is stimulated in a stimulation procedure in which at least one of the zona incerta, lenticular fasciculus, and motor region of the globus pallidus and internal capsule are stimulated, while current is not spread to, and therefore there is no stimulation of, any of the corticospinal tract (CS), corticobulbar tract (CB), and the non-motor regions of the globus pallidus (GP) and internal capsule.

For example, a target VTA can be created, for example, according to methods described in U.S. patent application Ser. No. 12/266,394, entitled “3D Atlas Fitting Using MicroElectrode Recordings” and filed Nov. 6, 2008, in U.S. Provisional Patent Application Ser. No. 61/120,006, entitled “System and Method to Define Target Volume for Stimulation in the Brain” and filed Dec. 4, 2008, in International Patent Application No. PCT/US09/066,821 entitled “System and Method to Define Target Volume for Stimulation in the Brain” and filed Dec. 4, 2009, in U.S. patent application Ser. No. 12/869,159, entitled “System and Method to Estimate Region of Tissue Activation” and filed Aug. 26, 2010, and in International Patent Application No. PCT/US1046772, entitled “System and Method to Estimate Region of Tissue Activation” and filed Aug. 26, 2010, the entire contents of each of which is hereby incorporated by reference in its entirety, such that they include no or minimal spread of current to the non-motor regions.

Example embodiments of the present invention are directed to systems and methods for selection of stimulation parameters for treatment of neuro-degenerative disorders, such as neuro-cognitive and/or neuro-motor disorders based on results of tests of cognitive function, tests of motor function, and a combination of such tests, performed by the patient to which stimulation is to be performed using the selected stimulation parameters. The stimulation can be performed using implanted electrodes. In an example embodiment, the test results can be used for selection of stimulation parameters that minimize creep of current to non-motor anatomic regions, e.g., of the brain. In an example embodiment, the n-Back test can be used as the test for testing cognitive function, a force-maintenance task can be used as the test for testing motor function, and a test where a patient is subjected to both the n-Back test and the force-maintenance task simultaneously can be used as the combination as a “dual task.”

In alternative example embodiments of the present invention, the motor and cognitive testing discussed throughout the present application can be performed using motor, cognitive, and/or motor-cognitive tests described in U.S. Provisional Pat. App. Ser. No. 61/262,662, filed Nov. 19, 2009 (“the '662 application”) and/or in International Pat. App. No. PCT/US10/57453, filed Nov. 19, 2010 (“the '453 application”), the entire contents of each of which is hereby incorporated by reference herein. Those tests can be administered and the test results captured and recorded, for example, as described in the '662 application and/or the '453 application.

Example embodiments of the present invention are directed to a computer system configured to determine stimulation parameters based on the above-described tests, e.g., to minimize creep of current to non-motor anatomical regions. That is, to determine stimulation parameters, the system can evaluate various parameter settings using objective and quantitative test that have cognitive and motor components.

Programming DBS devices for maximal clinical benefit and minimal side effects can be a difficult and time consuming process, requiring a highly trained and experienced individual to achieve desirable results (Hunka, K. et al. “Nursing time to program and assess deep brain stimulators in movement disorder patients,” J. Neurosci. Nurs. 37, 204-10 (2005); Moro, E. et al., “Subthalamic nucleus stimulation: improvements in outcome with reprogramming,” Arch. Neurol. 63, 1266-72 (2006) (hereinafter “Moro et al., 2006”), the entire contents of each of which is hereby incorporated by reference herein). While guidelines exist on stimulation parameter settings that are typically effective (Moro et al., 2002; Rizzone et al., 2001; Volkmann, J. et al., “Basic algorithms for the programming of deep brain stimulation in Parkinson's disease,” Mov. Disord. 21 Suppl. 14, S284-9 (2006), the entire contents of each of which is hereby incorporated by reference herein), these vary from patient to patient and it is not practical to clinically evaluate each of the thousands of stimulation parameter combinations that are possible in order to optimize DBS in each patient. As a result, the therapeutic benefits achieved with DBS are strongly dependent on the intuitive skill and experience of the clinician performing the programming (Moro et al., 2006) and the amount of time each programmer can allocate to that patient.

Rather than relying solely on intuition and experience, clinical DBS programming according to the present invention can be augmented with visualization of electrode location and theoretical calculation of an optimal VTA. Software technology can provide an initial starting point for the clinical programming process, thereby focusing patient testing on a select range of stimulation settings where an abbreviated version of the dual-task paradigm could be performed to evaluate cognitive and motor function. For example, the VTA can be visualized before the programming user even sees the patient. The programming user can essentially test a host of parameter sets using the software rather than having to actually apply those parameters to stimulation of the patient. The parameters that are most likely ineffective can therefore be eliminated to begin with. Additionally, the software itself can be programmed with parameter sets that are most likely ineffective due to, for example, current creep to non-motor regions, and can accordingly output suggested sets of parameter settings. The dual-task paradigm could concentrate clinical resources on maximizing clinical outcomes and minimize time consuming searches through the DBS parameter space (contact, voltage, pulse width, frequency).

According to an example embodiment of the present invention, cognitive and motor performance are evaluated simultaneously, e.g., by administering the above-described tests, during DBS programming while visualizing VTAs associated with specific DBS parameters. This can mitigate the described paradoxical situation between the clinical improvements in motor functioning and the patient and caregiver's level of postoperative satisfaction. That is, by modifying DBS parameters based on the test results, the patient satisfaction to the DBS programming using the resultant stimulation parameters can be consistent with the motor benefits.

In this regard, by visualizing the various VTAs while simultaneously assessing the cognitive and motor performance as described herein, the medical clinician can rank various VTAs according to such performance and modify the target VTAs until stimulation parameters are provided for a closely matching estimated VTA that produces the best cognitive and motor performance. The clinician can input notes in association with VTAs, which notes identify and/or rank the assessed cognitive and motor performance. The system can include a graphical user interface (GUI) that displays an anatomical model, e.g., of the patient brain and implanted leadwire, that further displays with respect to the displayed model one or more areas corresponding to explored VTAs, and that further displays note icons representing the input notes associated with such VTAs and displayed such that it is indicated with which VTAs the corresponding notes are associated. Responsive to selection of the note icons, the systems and methods can display the notes.

According to an example embodiment of the present invention, the clinician can input a score associated with the explored VTAs based on the cognitive and motor function, and for those VTAs for which a score not meeting a predetermined threshold, the system can treat the VTA as one associated with a side effect. The systems and methods can visually indicate which explored VTAs are associated with side effects and which are not. Such graphics can be further considered by the clinician to ultimately make a final selection of the stimulation parameters to use.

It is noted that other factors can be used in selection of stimulation parameters, and, while the assessed cognitive and motor performance can be considered, the parameters resulting in the very best performance are not necessarily selected. Accordingly, parameters which result in a good performance, but not necessarily the best, can be selected.

In an example, a system user can select initial parameter settings by inputting a target VTA with minimal current creep to the non-motor regions and obtaining settings that provide an estimated VTA closely matching the target VTA. If the patient performs poorly on the administered tests, a new target VTA can be drawn with even less creep to the non-motor regions or at further distance from such regions, etc. As noted above, the cognitive and motor performance can be tested while visualizing the VTAs associated with particular parameters. The clinician can keep tweaking the target VTAs according to the test performance. That is, the clinician will be able to quickly see the results of the performance and those results relate to the position and size of the VTA.

Example embodiment of the present invention are directed to a computer system configured to provide a GUI via which the computer system can obtain user input according to which the computer system is configured to output a representation of a target VTA. The user input can be stimulation parameters and/or references to anatomical points, e.g., of a perimeter of the target VTA. The computer system can be configured to further provide a GUI via which the user can adjust parameters or points of the target VTA to obtain a desired target VTA. For example, the target VTA can be one that avoids non-motor anatomical regions to the extent described above. In an example embodiment of the present invention, the system can determine stimulation parameter settings that are estimated to provide a VTA that most closely matches the input target VTA. In an example embodiment, the system can operate under a condition that it limits the most closely matching estimated VTA to one that does not protrude beyond any point of the outer perimeter of the target VTA (even though there may be such a VTA that is a closer match to the target VTA).

For obtaining input for the generation of, and for generating, the target VTAs and/or for determining estimated VTAs, and/or for recording and visually outputting notes or indications of notes concerning cognitive and motor performance associated with VTAs, the systems and methods of the present invention can, for example, use processes described in U.S. patent application Ser. No. 12/454,330, filed May 15, 2009 and entitled “Clinician Programmer System and Method for Calculating Volumes of Activation” (“the '330 application”), in U.S. patent application Ser. No. 12/454,312, filed May 15, 2009 and entitled “Clinician Programmer System and Method for Calculating Volumes of Activation for Monopolar and Bipolar Electrode Configurations” (“the '312 application”), in U.S. patent application Ser. No. 12/454,340, filed May 15, 2009 and entitled “Clinician Programmer System and Method for Steering Volumes of Activation” (“the '340 application”), in U.S. patent application Ser. No. 12/454,343, filed May 15, 2009 and entitled “Clinician Programmer System Interface for Monitoring Patient Progress” (“the '343 application”), and in U.S. patent application Ser. No. 12/454,314, filed May 15, 2009 and entitled “Clinician Programmer System and Method for Generating Interface Models and Displays of Volumes of Activation” (“the '314 application”), the content of each of which is hereby incorporated herein by reference in its entirety.

Example embodiments of the present invention are directed to a computer system configured to monitor a patient performance during, and/or to record results of, the tests described above, the results of which can be used to select the stimulation parameters.

The computer system(s) can include one or more processors, which can be implemented using any conventional processing circuit and device or combination thereof, e.g., a Central Processing Unit (CPU) of a Personal Computer (PC) or other workstation processor, to execute code provided, e.g., on a hardware computer-readable medium including any conventional memory device, to perform any of the methods described herein, alone or in combination. The one or more processors can be embodied in a server or user terminal or combination thereof. The user terminal can be embodied, for example, as a desktop, laptop, hand-held device, Personal Digital Assistant (PDA), television set-top Internet appliance, mobile telephone, smart phone, etc., or as a combination of one or more thereof. The memory device can include any conventional permanent and/or temporary memory circuits or combination thereof, a non-exhaustive list of which includes Random Access Memory (RAM), Read Only Memory (ROM), Compact Disks (CD), Digital Versatile Disk (DVD), and magnetic tape.

Example embodiments of the present invention are directed to one or more hardware computer-readable media, e.g., as described above, having stored thereon instructions executable by a processor to perform the methods described herein.

Example embodiments of the present invention are directed to a method, e.g., of a hardware component or machine, of transmitting instructions executable by a processor to perform the methods described herein.

Bilateral deep brain stimulation (DBS) of the subthalamic nucleus (STN) is an effective therapy for improving the cardinal motor signs of advanced Parkinson's disease (PD) (The Deep Brain Stimulation Study Group, “Deep-brain stimulation of the subthalamic nucleus or the pars interna of the globus pallidus in Parkinson's disease,” N. Engl. J. Med. 345, 956-63 (2001), the entire contents of which is hereby incorporated by reference herein). Other target sites are effective for treating other motor, cognitive, and/or cognitive-motor disorders as outlined above. While bilateral STN DBS is considered safe, an emerging concern is the potential negative consequences it may have on cognitive functioning and overall quality of life (Freund, H. J., “Long-term effects of deep brain stimulation in Parkinson's disease,” Brain 128, 2222-3 (2005); Rodriguez-Oroz et al., 2005; Saint-Cyr, J. A. et al., “Neuropsychological consequences of chronic bilateral stimulation of the subthalamic nucleus in Parkinson's disease,” Brain 123 (Pt 10), 2091-2108 (2000), the entire contents of each of which is hereby incorporated by reference herein). A recent report indicates patients' perceptions of their day-to-day function is improved subtly by DBS; however, caregivers perceived the patient as exhibiting subtle declines in day-to-day functioning (Duff-Canning, S. J. et al., “He said, she said: Differences between self and caregiver ratings of postoperative behavioral changes in Parkinson's disease patients undergoing bilateral subthalamic nucleus deep brain stimulation,” In: Twelfth International Congress of Parkinson's disease and Movement Disorders, vol. 23, ed.̂eds. Wiley-Blackwell, Chicago, Ill., p. S127 (2008), the entire contents of which is hereby incorporated by reference herein).

Several long-term studies examining changes in cognitive function suggest that bilateral STN DBS results in varying levels of decline in overall cognitive functioning, including verbal fluency (Contarino, M. F. et al., “Cognitive outcome 5 years after bilateral chronic stimulation of subthalamic nucleus in patients with Parkinson's disease,” J Neurol Neurosurg Psychiatry 78, 248-52 (2007); Funkiewiez, A. et al., “Long term effects of bilateral subthalamic nucleus stimulation on cognitive function, mood, and behaviour in Parkinson's disease,” J Neurol Neurosurg Psychiatry 75, 834-9 (2004), the contents of each of which is hereby incorporated by reference herein) and working memory (Rodriguez-Oroz et al., 2005; Schupbach, W. M. et al., “Stimulation of the subthalamic nucleus in Parkinson's disease: a 5 year follow up,” J. Neurol. Neurosurg. Psychiatry 76, 1640-4 (2005) (hereinafter “Schupbach et al., 2005”), the entire contents of each of which is hereby incorporated by reference herein). Although some of these long term results may be due to natural progression of PD, they provide compelling evidence to suggest that bilateral STN DBS may adversely affect different features of cognitive functioning and bring into question earlier views that STN DBS does not impair cognition. For example, measures of verbal fluency and learning and memory, exhibited significant declines when comparing bilateral STN DBS to pre-surgery or OFF DBS scores (OFF typically referring to the temporary turn off of DBS for a research or clinical protocol) (Woods, S. P. et al., “Neuropsychological sequelae of subthalamic nucleus deep brain stimulation in Parkinson's disease: a critical review,” Neuropsychol. Rev. 12, 111-2642002), the entire contents of which is hereby incorporated by reference herein). In a meta-analysis that included data from 1,398 patients with bilateral STN DBS, cognitive problems were seen in 41 percent of patients (Temel, Y. et al., “Behavioural changes after bilateral subthalamic stimulation in advanced Parkinson disease: A systematic review,” Parkinsonism Relat. Disord. (2006), the entire contents of which is hereby incorporated by reference herein). Cognitive problems varied from a moderate deterioration in verbal memory to significant declines in executive functioning.

While cognitive declines are commonly seen with STN DBS, the degree of measured effect may be attributable to variation in the difficulty of the cognitive testing across studies Hershey, T. et al., “Stimulation of STN impairs aspects of cognitive control in PD,” Neurology 62, 1110-4 (2004), the entire contents of which is hereby incorporated by reference herein). The majority of studies examining the cognitive effects of STN DBS have utilized relatively simple neuropsychological tests suitable for use in a clinical environment. Therefore, reports of no or minimal effect of STN DBS on cognitive functioning may be explained by a lack of difficulty in test selection or the artificial environmental, free of distraction, in which they are completed. Hershey and colleagues (Hershey et al., 2004) reported that bilateral STN stimulation decreased working memory under cognitively demanding conditions. Those results are added to by examining cognitive and motor function individually and simultaneously under different levels of cognitive demands (Alberts et al., 2008). As working memory demands increased, cognitive, motor and cognitive-motor function decreased during bilateral compared to unilateral STN DBS (Alberts et al., 2008). Based on the results described herein, it is believed that the spread of current to non-motor regions of each STN may be responsible for the disruption in cognitive, motor and cognitive-motor function during bilateral STN DBS.

Given its small size, stimulation within the STN, even with electrode contacts located predominately within the sensorimotor territory, can result in the spread of current to limbic and associative areas as well as to surrounding structures and fiber systems that can also affect cognition (Maks, C. B. et al., “Deep brain stimulation activation volumes and their association with neurophysiological mapping and therapeutic outcomes,” J. Neurol. Neurosurg. Psychiatry 80, 659-66 (2009), the entire contents of which is hereby incorporated by reference herein). The electric field generated by DBS is non-discriminately applied to all of the neural elements surrounding the electrode, and these stimulation effects are subsequently transmitted throughout the basal ganglia and thalamocortical networks (Asanuma, K. et al., “Network modulation in the treatment of Parkinson's disease,” Brain 129, 2667-78 (2006); Karimi, M. et al., “Subthalamic nucleus stimulation-induced regional blood flow responses correlate with improvement of motor signs in Parkinson disease,” Brain 131, 2710-9 (2008); Phillips, M. D. et al., “Parkinson disease: pattern of functional MR imaging activation during deep brain stimulation of subthalamic nucleus—initial experience,” Radiology 239, 209-16 (2006), the entire contents of each of which is hereby incorporated by reference herein). In turn, diminished cognitive function may be due to nonselective activation of non-motor pathways within and around the STN. However, according to the present invention, when the STN is stimulated, current spread to limbic and associative regions as well as throughout the basal ganglia and Thalamocortical networks is avoided through software modeling and calculations of those VTAs.

The interplay between the patient and clinician performing the DBS parameter selection is critical in defining the balance between therapeutic benefit and stimulation induced side effects. However, clinical DBS programming is typically done without the opportunity to visualize the spread of stimulation relative to the surrounding anatomy. In turn, current spread into non-target areas could occur without overt clinical signs, but still result in side effects not typically tested for in traditional clinical programming sessions. Therefore, recently developed Windows-based software tools that enable 3D visualization of the volume of tissue activated (VTA) by DBS as a function of the stimulation parameters and electrode location in the brain have been developed (Butson, C. R. et al., “StimExplorer: deep brain stimulation parameter selection software system,” Acta Neurochir Suppl. 97, 569-74 (2007) (hereinafter “Butson et al., 2007b”); Miocinovic et al., 2007, the entire contents of each of which is hereby incorporated by reference herein). In an example embodiment, quantitative theoretical predictions are used to define stimulation parameter settings, customized to the patient, maximizing stimulation of target areas and minimizing stimulation spread to non-target areas.

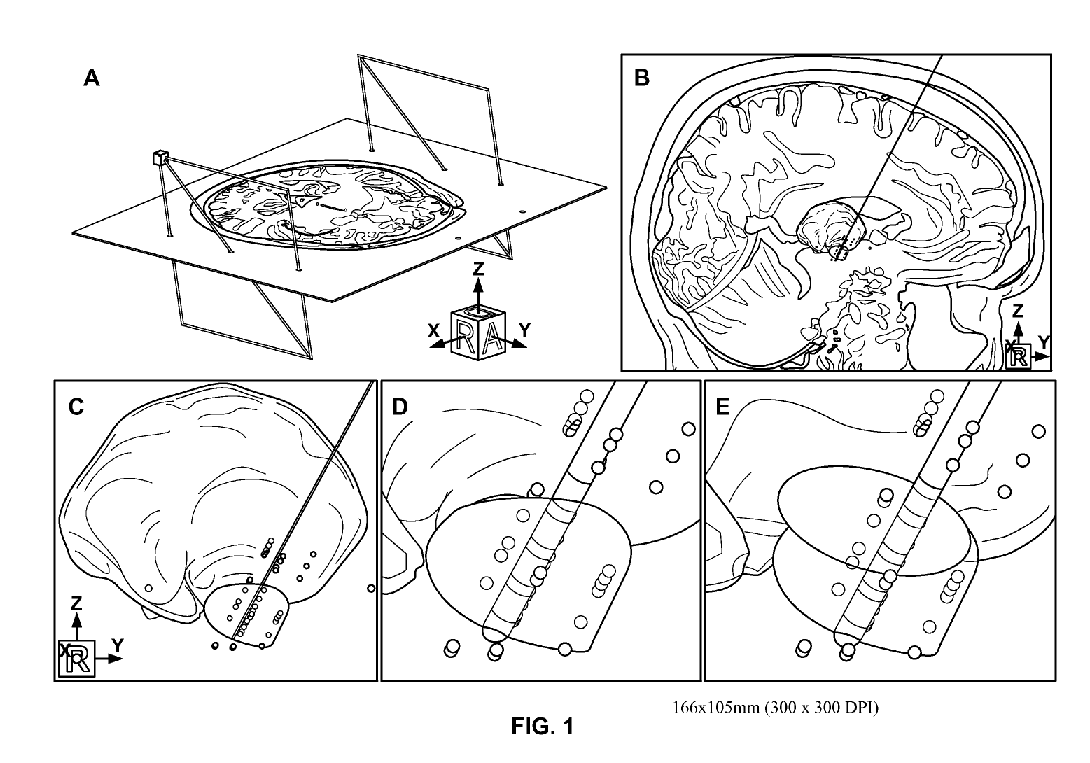

Described herein is a comparison of the effectiveness of two DBS programming strategies, standard Clinical (where current is spread throughout the dorsal and ventral portions of the STN) and Model-based, on cognitive-motor performance in advanced PD patients under dual-task conditions, where the primary criterion for the selection of Model DBS parameters is maximizing stimulation of target areas in the subthalamic region while minimizing stimulation of associative/limbic (ventral) sections of the STN. The target areas were defined as the dorsal STN and white matter dorsal to the STN (FIG. 1) (Butson, C. R. et al., “Patient-specific analysis of the volume of tissue activated during deep brain stimulation,” Neuroimage 34, 661-70 (2007) (hereinafter “Butson et al., 2007a”); Maks et al., 2009). Minimizing spread of current to the non-motor regions of the STN and focusing current spread to areas previously shown to produce ideal therapeutic benefit can minimize cognitive-motor declines under dual-task conditions without compromising improvements in motor function.

According to an example embodiment of the present invention, a computer-implemented method includes selecting, by a computer processor, stimulation parameters for stimulating an anatomical region of a patient, the selection being based on results of at least one dual task test that tests cognitive and motor functions, where the motor functions include postural stability.

In an example embodiment, the stimulation parameters are selected for treatment of a neuro-degenerative disorder, and the at least one dual task test is conducted on the patient to be stimulated using the selected stimulation parameters.

In an example embodiment, the stimulation is performed using implanted electrodes of a deep brain stimulation (DBS) device.

In an example embodiment, the method further includes calculating, in conjunction with the at least one dual task test, a computational model of estimated activated tissue for previously selected stimulation parameters to optimize the selecting of the stimulation parameters.

In an example embodiment, the computational model provides a visualization of an estimated volume of activated tissue relative to patient-specific anatomical images and the implanted electrodes.

In an example embodiment, the computational model calculates an estimated volume of activated tissue for the selected stimulation parameters, and new stimulation parameters are selected based on the calculated estimated volume of activated tissue, which new parameters are predicted to result in a modified region of activated tissue that includes (i) a target motor anatomical region and (ii) minimization of current creep to a non-motor anatomical region as compared to the calculated estimated volume of activated tissue.

In an example embodiment, the motor function task includes the patient standing upright on a force platform while fixating at a distant target.

In an example embodiment, the method further includes calculating displacement and center of pressure data as results of the motor function task; and evaluating the data via a best fit ellipse to determine the patient's postural sway.

In an example embodiment, the cognitive function task is an n-Back task used for evaluating working memory, the motor function task is used for evaluating postural stability, and the patient is subjected to both the n-Back task and the postural stability task simultaneously.

In an example embodiment, the selection is performed to maximize stimulation of a target motor anatomical region and minimize current creep to non-motor anatomical regions.

In an example embodiment, the anatomical regions are part of the subthalamic nucleus (STN).

In an example embodiment, the results of the at least one dual task test are used to optimize the stimulation parameters to minimize the current creep to the non-motor anatomical region.

In an example embodiment, the stimulation parameters are selected to minimize stimulation-related cognitive declines.

In an example embodiment, the dual task test includes performance of a motor function task used to evaluate postural sway.

In an example embodiment, the parameters are selected to improve postural stability.

In an example embodiment, the parameters are selected to improve cognitive function.

In an example embodiment, the parameters are selected to improve both cognitive function and postural stability.

In an example embodiment, the at least one dual task test includes a plurality of dual task tests.

According to an example embodiment of the present invention, a computer-implemented method includes assessing, by a computer processor, a previously performed stimulation of an anatomical region of a patient by analyzing results of at least one dual task test that tests cognitive and motor functions, where the motor functions include postural stability.

According to an example embodiment of the present invention, a computer-readable medium has stored thereon instructions executable by a processor, the instructions which, when executed by the processor, cause the processor to perform a method, the method including selecting stimulation parameters for stimulating an anatomical region of a patient, the selection being based on results of at least one dual task test that tests cognitive and motor functions, where the motor functions include postural stability.

According to an example embodiment of the present invention, a system includes a computer processor configured to select stimulation parameters for stimulating an anatomcal region of a patient, the selection being based on results of at least one dual task test that tests cognitive and motor functions, where the motor functions include postural stability.

### First Study

A total of 10 participants with advanced PD between the ages of 51 and 72 years (mean 58.6) participated in a study. Table 1 contains patient demographics and time since DBS surgery (DBS duration) and Table 2 contains Clinical and Model DBS parameters. All patients had undergone simultaneous bilateral STN DBS surgery at the Cleveland Clinic at least 14 months prior to study participation. Surgical procedures for DBS implantation have been reported in detail previously (Machado, A. et al., “Deep brain stimulation for Parkinson's, disease: surgical technique and perioperative management,” Mov. Disord. 21 Suppl. 14, S247-58 (2006), the entire contents of which is hereby incorporated by reference herein). Stimulation parameters for DBS devices were clinically determined using the methods described by Moro and colleagues (Moro et al., 2006) and were stable for at least six months prior to study participation. The programming of stimulators was overseen by an experienced DBS programming team consisting of a programming nurse and movement disorders neurologist specializing in PD. Because participants needed to make verbal responses during the working memory test, patients with dysarthria or speech impairment were excluded. Prior to data collection, all participants signed an informed consent approved by the Cleveland Clinic Institutional Review Board.

TABLE 1
Patient demographics and UPDRS-III scores during Off, Clinical and Model DBS
conditions and the percent change from Off to Clinical and Off to Model DBS.

| | | | DBS | | | | UPDRS-III (%) | |
|---|---|---|---|---|---|---|---|---|
| | | Age | duration | UPDRS-III score | | | Off to | Off to |
| Patient | Gender | (years) | (months) | Off | Clinical | Model | Clinical | Model |
| 1 | F | 52 | 14 | 61 | 32 | 35 | 47.54 | 42.62 |
| 2 | M | 51 | 40 | 65 | 30 | 40 | 53.85 | 38.46 |
| 3 | M | 54 | 26 | 50 | 31 | 31 | 38.00 | 38.00 |
| 4 | M | 63 | 38 | 56 | 35 | 32 | 37.50 | 42.86 |
| 5 | M | 71 | 29 | 61 | 26 | 30 | 57.38 | 50.82 |
| 6 | M | 53 | 17 | 44 | 26 | 18 | 40.91 | 59.09 |
| 7 | M | 72 | 35 | 51 | 31 | 29 | 39.22 | 43.14 |
| 8 | M | 51 | 33 | 55 | 30 | 27 | 45.45 | 50.91 |
| 9 | M | 61 | 45 | 68 | 28 | 31 | 58.82 | 54.41 |
| 10 | M | 58 | 14 | 56 | 31 | 32 | 44.64 | 42.86 |
| Mean | | 58.60 | 29.10 | 56.70 | 30.00 | 30.50 | 46.33 | 46.32 |
| SD | | 7.55 | 10.55 | 6.90 | 2.61 | 5.35 | 7.53 | 6.70 |

A 6 degree of freedom force-torque transducer (Mini-40 Model, ATI Industrial Automation, Garner, N.C., USA) was used to measure normal force (Fz; grip) during a force-tracking motor task. Grip force was measured with a resolution of 0.06 N at a sampling rate of 128 Hz. A customized LabView program developed by the inventors' laboratory was used to collect and display force data to the participant. In an example embodiment of the present invention, stimulation parameters can be selected based on results of a force-maintenance task test (e.g., in combination with a cognitive function test), where the force-maintenance task test is performed using a 6 degree of freedom force-torque transducer to measure the force. Moreover, the described resolution of 0.06 N can be used at the sampling rate of 128 Hz.

### The N-Back Task

Various forms of the n-back task have been used in a number of previous studies (for comprehensive review see Owen, A. M. et al., “N-back working memory paradigm: a meta-analysis of normative functional neuroimaging studies,” Hum. Brain Mapp. 25, 46-59 (2005), the entire contents of which is hereby incorporated by reference herein). The n-back task utilized in the current study was based on the methods originally used in its development. This version of the n-back task requires the participant to repeat the nth item back (e.g., O-back, 1-back, 2-back) in a sequentially presented list of items (Dobbs, A. R. et al., “Adult age differences in working memory,” Psychol Aging 4, 500-3 (1989), the entire contents of which is hereby incorporated by reference herein). This same technique was used in a recent dual-task study with advanced PD patients during unilateral and bilateral STN DBS (Alberts et al., 2008). The difficulty level of the n-back task is manipulated by requiring the participants to remember items further back in the list. The type of n-back test used in this study utilized a list of random letters presented to the participant. The number of intervening letters varied from zero to two. This method of n-back testing requires encoding, maintenance, updating and output. However, unlike other versions of the task it does not require comparison or decision-making.

Two English-speaking experimenters administered the n-back task. Experimenter 1 read aloud the randomized letter sets of the n-back task while experimenter 2 monitored the participant's responses for accuracy. Participants were asked to respond by articulating the letter presented directly before (0-back), 1 cycle before (1-back), or two cycles before (2-back). If the participant provided an incorrect response or was unable to answer correctly within the allotted time (1.5 s) the trial would begin with a new sequence of letters. If the participant provides the correct answer, additional letters can be presented for the rest of the 30 second trial. Approximately 19-23 trials (letters) were presented during a 30 second block. After performing the n-back task for 30 seconds, participants rested for 15-45 seconds and then repeated the n-back task under the same level of difficulty (0, 1- or 2-back). Participants performed five 30 second blocks at each n-back condition (0, 1- and 2-back). These five blocks were collected sequentially and were randomized across participants. To account for practice effects, all participants completed three practice trials (30 seconds each) at each n-back level prior to data collection. Three trials have been shown to be sufficient to ensure task comprehension and stable performance for advanced PD patients (Alberts et al., 2008); all participants in the current study reported task comprehension and demonstrated stable performance. All practice and test blocks consisted of a unique list of randomized letters to prevent any memorization of letters. In an embodiment in which parameters are selected on a per-patient basis based on how the patient performs during the described tests, the n-back test can be administered and parameter selection can be based on number of total errors during 30 seconds, number of correct responses, and number of letters before the first error.

### Force-Maintenance Task

Participants used a precision grip (i.e., thumb and index finger only) of their dominant hand to exert an isometric force against the force transducer. Similarly, in an example embodiment of the present invention, a precision grip can be used in a force maintenance test, based on results of which stimulation parameters are selected. The participant's dominant hand was determined using the Edinburg Handedness Inventory (Oldfield, R. C., “The assessment and analysis of handedness: the Edinburgh inventory,” Neuropsychologia. 9, 97-113 (1971), the entire contents of each of which is hereby incorporated by reference herein). The force transducer was oriented in a comfortable position to the patient and affixed to the table to prevent any movement and for consistency throughout force tracking. Three maximum precision grip efforts, 5 seconds each, were completed at each of the three data collection sessions. These data were used to establish the maximum grip force of the patient. Between each maximum effort, patients rested 1-2 minutes. The peak force achieved from the three efforts was considered the maximum and was used to calculate a target force level; 20% of the maximum force. A target force level can similarly be selected for administering a test based on which to select stimulation parameters for a patient who performs the test. The 20% target force level was selected as Galganski and colleagues (Galganski, M. E. et al., “Reduced control of motor output in a human hand muscle of elderly subjects during submaximal contractions,” J. Neurophysiol. 69, 2108-2115 (1993), the entire contents of which is hereby incorporated by reference herein) found no differences in younger adults' and older adults' standard deviation (SD) at this force level and based on previous studies with younger adults, older adults and advanced PD patients this force level could be maintained relatively easily with minimal fatigue (Alberts et al., 2008; Voelcker-Rehage, C., Alberts, J. L., “Age-related difference in working memory and force control under dual-task conditions,” Aging, Neuropsychology, and Cognition 13, 1-19 (2006) (hereinafter “Voelcker-Rehage and Alberts, 2006”); Voelcker-Rehage, C. et al., “Effect of motor practice on dual-task performance in older adults,” J. Gerontol B Psychol. Sci. Soc. Sci. 62, P141-8 (2007) (hereinafter “Voelcker-Rehage and Alberts, 2007”), the entire contents of each of which is hereby incorporated by reference herein). The target force level produced and actual real-time grip force produced by the participant was displayed on a 21″ LCD monitor located ˜44-59 cm directly in front of the participant. Participants were instructed to match their grip force to the target force line as accurately as possible. An auditory stimulus “ready, go” signaled the participants to start matching their force to the target force. Participants performed one to five practice repetitions prior to test blocks to be certain all task requirements were understood. Ten force-maintenance blocks for each limb, 30 seconds each, were performed with at least 30 seconds of rest between each block. The test administered to a patient for determining stimulation parameters for the patient, according to example embodiments of the present invention, can be similarly administered.

### Dual Task: N-Back and Force Maintenance Simultaneously

Participants performed 15 dual-task blocks in which they were asked to simultaneously perform the n-back task and force maintenance task. The force maintenance task was performed in random combination with each of the three n-back conditions (0-back, 1-back, 2-back; five repetitions each). Participants were instructed to perform both tasks as accurately as possible and to devote half of their attention to the cognitive task and half of their attention to the motor task. Participants were given at least 30 seconds of rest between each block. The tests for selection of stimulation parameters on a per patient basis can be similarly administered.

### Selection of Model DBS Parameters

For each subject enrolled in the study a patient-specific DBS computer model of each side of the patient's brain using Cicerone v1.2, a freely available academic DBS research tool (Miocinovic et al., 2007) (FIG. 1) was created. The models were created without any a priori knowledge of the patient, aside from access to their clinical MRI and CT imaging data, surgical targeting data, and intra-operative microelectrode recording (MER) data. Researchers were blinded to each patient's clinical symptoms, drug regiment, clinical DBS programming notes, and Clinical stimulation parameter settings.

Each patient-specific DBS model included coupled integration of MRI/CT data, MER data, 3D brain atlas surfaces, DBS electrodes, and VTA predictions all co-registered into the neurosurgical stereotactic coordinate system following previously described methodology (FIG. 1) (Butson et al., 2007a; Butson et al., 2007b; Miocinovic et al., 2007, all of which are incorporated by reference herein). The first phase of model development was to import imaging data into the software. The stereotactic coordinate system was defined by identifying fiducial landmarks of the neurosurgical head frame used to implant the electrode (FIG. 1A). The CT or MRI acquired with the frame in place was called the frame image and any subsequent imaging data used in the model was co-registered to the frame image. Co-registration between the frame image and an alternative image was performed manually within Cicerone using a two step process. First, coordinates of the anterior and posterior commissures (AC/PC), defined by the operating neurosurgeon, were used to initially register the two images together. Second, a nine panel graphical user interface (GUI) allowed for manual manipulation to fine tune the image fusion. This GUI displayed the axial, coronal, and saggital views of the frame image on the left column, the alternative image on the right column and an overlay of the two in the middle column. Because the images were from the same individual a rigid body transformation could be performed to bring the images into near perfect alignment.

The second phase of model development consisted of entering the stereotactic location of each MER data point, color coded based on its neurophysiologically defined nucleus, into the model (FIG. 1B,C). 3D anatomical representations of the various nuclei of interest (thalamus, subthalamic nucleus, etc.) were then scaled and positioned within the context of the pre-operative MRI and MER data (FIG. 1B,C). This process was performed manually, taking into account both anatomical structures visible in the MRI and fitting MER points within their respective nuclei, to provide the best possible overall fit of the brain atlas to the patient (Lujan, J. L. et al., “Automated 3-Dimensional Brain Atlas Fitting to Microelectrode Recordings from Deep Brain Stimulation Surgeries,” Stereotact. Funct. Neurosurg. 87, 229-240 (2009); Maks et al., 2009). Once the patient's anatomical model was defined, the electrode type (Medtronic Electrode Model 3387 or 3389) was selected and the implantation position of the DBS electrode, as defined by intra-operative stereotactic coordinates, was displayed within the model system (FIG. 1D). Comparison with the post-operative CT verified that the intended surgical placement of the DBS electrode was within the artifact of the imaged electrode.

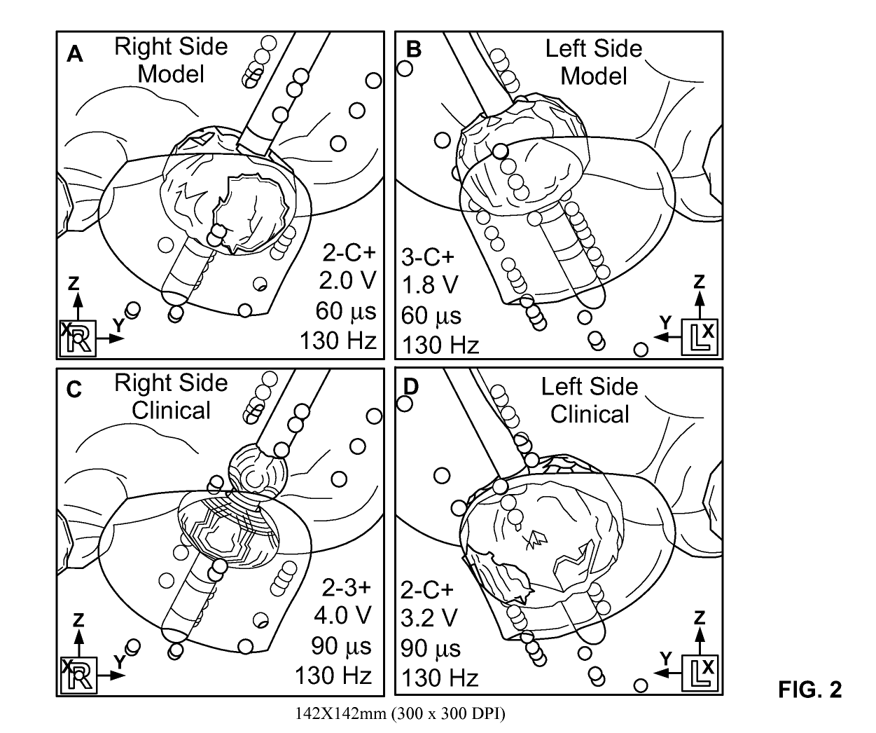

Based on previous experience developing patient-specific models of therapeutic STN DBS (Butson et al., 2007a; Maks et al., 2009), a theoretical ellipsoid target volume (FIG. 1E) was defined. Stimulation of this target area, which included the dorsal STN and white matter dorsal to the STN, has been associated with excellent clinical outcomes in previous work. A stimulation parameter setting was defined for each side of each patient's brain that maximized stimulation coverage of the target volume and minimized stimulation spread outside of the target volume. This theoretically optimal parameter setting was called the “Model DBS” and it was defined using theoretical predictions of the volume of tissue activated (VTA) (FIG. 2). The VTA provides an electrical prediction of the volume of axonal tissue directly activated by DBS for a given stimulation parameter setting. The VTAs used in Cicerone v1.2 are pre-compiled solutions from the DBS models previously described. (Butson, C. R. et al., “Predicting the effects of deep brain stimulation with diffusion tensor based electric field models,” Medical Image Computing and Computer Assisted Intervention, International Conference on Medical Image Computing and Computer Assisted Intervention 9, 429-37 (2006) (hereinafter “Butson et al., 2006”), the entire contents of which is hereby incorporated by reference). The software provided the ability to quickly and interactively evaluate a wide range of stimulation parameter settings and enable definition of a theoretically optimal Model DBS for each side of each patient (Table 2).

TABLE 2
Clinical and model stimulation parameters for all patients

| | Clinical Settings | | | | Model Settings | | | |
|---|---|---|---|---|---|---|---|---|
| | | | Pulse | | | | Pulse | |
| | | Voltage | Width | Frequency | | Voltage | Width | Frequency |
| Patient | Contact | (V) | (μs) | (Hz) | Contact | (V) | (μs) | (Hz) |

Left Stimulation Parameters

| | | | | | | | | |
|---|---|---|---|---|---|---|---|---|
| 1 | 2-C+ | 3.2 | 90 | 130 | 3-C+ | 1.8 | 60 | 130 |
| 2 | 2-C+ | 3.2 | 90 | 185 | 2-C+ | 2.6 | 60 | 130 |
| 3 | 2-3+ | 3.5 | 60 | 135 | 2-C+ | 2.3 | 60 | 130 |
| 4 | 2-3+ | 3.6 | 60 | 135 | 2-C+ | 1.8 | 60 | 130 |
| 5 | 2-3+ | 3.6 | 90 | 135 | 2-C+ | 2.6 | 60 | 130 |
| 6 | 2-C+ | 3.0 | 60 | 130 | 2-C+ | 2.4 | 60 | 130 |
| 7 | 1-3+ | 3.6 | 90 | 185 | 2-C+ | 2.5 | 60 | 130 |
| 8 | 1-C+ | 3.2 | 90 | 135 | 2-C+ | 2.4 | 60 | 130 |
| 9 | 1-2-C+ | 2.9 | 60 | 130 | 2-C+ | 1.8 | 60 | 130 |
| 10 | 1-C+ | 3.2 | 60 | 185 | 2-C+ | 2.4 | 60 | 130 |

Right Stimulation Parameters

| | | | | | | | | |
|---|---|---|---|---|---|---|---|---|
| 1 | 2-3+ | 4.0 | 90 | 130 | 2-C+ | 2.0 | 60 | 130 |
| 2 | 1-C+ | 3.6 | 60 | 185 | 2-C+ | 2.2 | 60 | 130 |
| 3 | 1-2+ | 3.5 | 60 | 135 | 2-C+ | 2.6 | 60 | 130 |
| 4 | 1-3+ | 3.3 | 60 | 135 | 2-C+ | 2.8 | 60 | 130 |
| 5 | 2-3+ | 3.9 | 90 | 135 | 2-C+ | 2.8 | 60 | 130 |
| 6 | 2-C+ | 3.2 | 60 | 130 | 2-C+ | 2.6 | 60 | 130 |
| 7 | 2-C+ | 3.6 | 90 | 185 | 3-C+ | 1.5 | 60 | 130 |
| 8 | 2-C+ | 3.2 | 60 | 135 | 2-C+ | 1.8 | 60 | 130 |
| 9 | 1-2-C+ | 2.9 | 60 | 130 | 2-C+ | 2.4 | 60 | 130 |
| 10 | 2-C+ | 3.2 | 60 | 185 | 2-C+ | 2.0 | 60 | 130 |

Following completion of the clinical study, the VTAs for each patient were quantified under both the Model and Clinical settings, along with their respective overlap with the STN volume. Each STN volume, as fitted to each hemisphere of each patient, was divided into a ventral and dorsal section. The STN division was defined by a plane parallel to the AC/PC plane that cut through the centroid of the STN. Table 3 contains the total VTA for each DBS condition and the percent in the ventral and dorsal portions of the STN (remaining numbers being outside the dorsal and ventral portions).

TABLE 3
Total volume of tissue activated (VTA) during Model and Clinical
DBS and the percent of VTA within the dorsal and ventral portions
of the STN for Model and Clinical settings.

| | | Model | | | Clinical | | |
|---|---|---|---|---|---|---|---|
| Patient | Side | total VTA | dorsal | ventral | total VTA | dorsal | ventral |
| 1 | Left | 45 | 13.8 | 0 | 116.6 | 47.4 | 18.9 |
| 1 | Right | 55.1 | 36.2 | 0.5 | 39 | 23.9 | 0 |
| 2 | Left | 71.7 | 20.3 | 0 | 108.9 | 31 | 0.6 |
| 2 | Right | 57.2 | 6.8 | 0 | 124.9 | 34.8 | 36.2 |
| 3 | Left | 65.4 | 28.2 | 2.3 | 29.1 | 12.3 | 0 |
| 3 | Right | 76.3 | 24.3 | 0.4 | 39.6 | 10.7 | 8.7 |
| 4 | Left | 49.6 | 25.7 | 0.3 | 29.8 | 15 | 0 |
| 4 | Right | 83.8 | 46.2 | 3.7 | 44.9 | 22.4 | 12.4 |
| 5 | Lett | 76.4 | 38.9 | 6.4 | 33.6 | 19.5 | 0.2 |
| 5 | Right | 84.1 | 45.2 | 2.4 | 37.4 | 20.5 | 0 |
| 6 | Left | 68.4 | 22.1 | 0 | 96.9 | 32.3 | 0 |
| 6 | Right | 73.7 | 27.4 | 0.4 | 106.3 | 37.2 | 2.3 |
| 7 | Left | 68.9 | 13 | 0 | 45.3 | 4.2 | 6.3 |
| 7 | Right | 35.4 | 25.3 | 3.3 | 137.2 | 35.7 | 35.7 |
| 8 | Left | 68.2 | 17.5 | 0 | 129.1 | 28.2 | 28.5 |
| 8 | Right | 49.5 | 25.5 | 1.7 | 103.7 | 41.3 | 9.5 |
| 9 | Left | 47.4 | 12 | 0 | 199.9 | 78.8 | 33 |
| 9 | Right | 65.5 | 25.8 | 0 | 199.9 | 68.6 | 43.6 |
| 10 | Left | 64.5 | 21 | 0 | 106.3 | 39.5 | 37.7 |
| 10 | Right | 52.9 | 13 | 0 | 96.3 | 25.4 | 0 |
| AVERAGE | | 63.0 | 24.4 | 1.1 | 91.2 | 31.4 | 13.7 |
| STDEV | | 13.4 | 10.8 | 1.7 | 53.0 | 18.3 | 15.9 |
| VTA (mm\hat{ }3) | | | | | | | |

### Calculation of Power Requirements for Stimulation Parameters

Waveforms were simulated according to the specific output of the Medtronic implanted pulse generator (Butson, C. R. et al., “Differences among implanted pulse generator waveforms cause variations in the neural response to deep brain stimulation,” Clin Neurophysiol. 118, 1889-94 (2007) (hereinafter “Butson and McIntyre, 2007”), the entire contents of which is hereby incorporated by reference herein). The power of stimulation with a given frequency, pulse width, and amplitude was calculated by averaging the instantaneous power over a 1 second period,

$${P}_{ta} = \frac{1}{T} {\int }_{0}^{T} \frac{{V ( t )}^{2}}{R} \cdot d t ,$$

where Pta is the time-averaged power, T is set to 1 s, V(t) is the instantaneous voltage, R input resistance, and t is time. The power consumption, in microwatts, was calculated for Clinical and Model DBS settings.

### Procedure

All data were collected during two visits to a research laboratory at the Cleveland Clinic. These two data collection sessions were separated by at least 72 hours. For both sessions, participants reported to the laboratory in the clinically defined off condition (i.e., at least 12 hours since their last dose of antiparkinsonian medication) while on DBS with their clinically defined stimulation parameters. After completing the informed consent process, patients were evaluated clinically with the Unified Parkinson's Disease Rating Scale (UPDRS) Part-III Motor Exam administered by an experienced movement disorders neurologist. The same neurologist completed all ratings except for one experimental session (patient 9; Clinical settings).

Each participant completed evaluation and testing under three DBS conditions: Off DBS, Clinical DBS, and Model DBS across the two laboratory visits. The order of testing Clinical and Model DBS parameters were randomized across patients across the two laboratory visits. For example, Day 1 testing consisted of completing all tests while on Clinical DBS and then following completion the patient's stimulator was turned Off for three hours and all clinical, motor, cognitive and cognitive-motor testing was repeated. On Day 2 the patient would complete all testing using the Model DBS parameters. Five patients were tested under Clinical DBS on Day 1 and five patients completed Model DBS on Day 1. Within each experimental session, the single task conditions were completed before the dual-task conditions. The single task conditions were the n-back task (three levels of difficulty: 0-1- and 2-back) and force maintenance task only. The order of completing the single task cognitive and motor tasks was randomized across patients. The order of dual-task conditions, force maintenance with the three different levels of n-back, was randomized across patients.

According to the embodiment where stimulation parameters are selected on a patient-specific basis based on results of such tests, the tests can be performed initially under the stimulation settings of the predicted model parameters as discussed above. Subsequently, the tests can be performed under parameters selected based on the clinician's judgment in view of the patient's performance on prior iterations of test administration and VTA size and shape for various settings. Additionally, the tests can be administered and data can be collected prior to programming when the patient has yet to have any stimulation, to obtain a baseline of cognitive-motor function.

The Clinical DBS and Off DBS experimental session patients completed all testing on two occasions within the same day: first under Clinical DBS parameters and then while Off DBS. After completing all clinical, cognitive, motor and cognitive-motor tests under Clinical DBS, the patient's stimulators were turned Off for three hours to allow the effects of DBS to wear off (Alberts, J. L. et al., “Comparison of pallidal and subthalamic stimulation on force control in patient's with Parkinson's disease,” Motor Control. 8, 484-99 (2004) (hereinafter “Alberts et al., 2004”); Alberts et al., 2008; Temperli, P. et al., “How do parkinsonian signs return after discontinuation of subthalamic DBS?,” Neurolog. 60, 78-81 (2003), the entire contents of each of which is hereby incorporated by reference herein). During this three hour wash out period the patient remained in the laboratory and was provided lunch and rested. Following the 3 hour wash out period, the patient repeated all clinical, cognitive, motor and cognitive-motor tests. Upon completion of this experimental session, the patient's stimulators were turned on (Clinical DBS parameters were restored) and they resumed their antiparkinsonian medication. Approximately 30 minutes after taking their medication and restoration of DBS the patient departed the laboratory. The total time spent in the laboratory during a Clinical DBS and Off DBS experimental session was approximately 5-6 hours (−2 hours of data collection and 3 hours rest during the wash out period).

The Model DBS experimental session, which randomly occurred on Day 1 or Day 2, was completed in approximately 4-5 hours. For the Model DBS session, the patients arrived in the laboratory off antiparkinsonian medication and on Clinical DBS. Upon arrival, both stimulators were turned Off. The patient then rested in the laboratory for the next two hours. After two hours the patient was re-programmed using the Model DBS parameters. After 60 minutes under Model DBS parameters, the patient completed all clinical, cognitive, motor and cognitive-motor testing. Upon completion of the Model DBS testing session, the patient's stimulators were reprogrammed to their clinically defined parameters and they took their anti-parkinsonian medication and departed the lab approximately 30 minutes later.

### Data Analysis

Force-maintenance: All force data were filtered with a phase-symmetric low-pass filter employing Woltring's algorithm (detailed in previous studies (Voelcker-Rehage, C., Stronge, A. J. et al., “Age-related differences in working memory and force control under dual-task conditions,” Neuropsychol. Dev. Cogn. B Aging Neuropsychol. Cogn. 13, 366-84 (2006) (hereinafter “Voelcker-Rehage et al., 2006”); Voelcker-Rehage and Alberts, 2007)) using existing Matlab analysis programs developed in the inventors' laboratory. Force data were assessed to determine the patients' accuracy from three seconds after the start of the block until completion of the block; this period allowed the patient sufficient time to achieve the target force. That is, test results were collected beginning after three seconds. The primary motor outcome variables for the force-tracking task were time within the target range (TWR) and relative root mean square error (RRMSE). The TWR is calculated by determining the time the patient's force trace is within ±2.5% of the target line, i.e. within 2.5% of the force, such that, for example, if the target force is 5N, the TWR is the time at which a force is maintained in the range of 4.375-5.625N. This can be different for each patient, based on the patient's target force. The assessment was done after the data collection so the patient was not targeting this region specifically. The TWR provides an overall accuracy measure of force-tracking. To account for differences in the amplitude of the target force (e.g., inter-patient and intra-patient variability due to stimulation status), the RRMSE, as defined in equation 1, was used as a method of normalizing performance relative to force amplitude. The RRMSE is considered to reflect the overall variability of force-tracking performance; a lower RRMSE suggests control of distal musculature and hand functionality (Kriz, G. et al., “Feedback-based training of grip force control in patients with brain damage,” Arch. Phys. Med. Rehabil. 76, 653-659 (1995); Kurillo, G. et al., “Force tracking system for the assessment of grip force control in patients with neuromuscular diseases,” Clin. Biomech (Bristol, Avon) 19, 1014-21 (2004), the entire contents of each of which is hereby incorporated by reference herein). In the equation below, F_{T}(t) is the target force provided to the patient, F₀(t) is the force produced by the patient and T is the time of the block.

$$R R M S E = \sqrt{\frac{1}{T} {\sum }_{t = 0}^{T} \frac{{( {F}_{0} ( t ) - {F}_{T} ( t ) )}^{2}}{{max ( {F}_{T} )}^{2}}}$$

TWR and RRMSE can be used according to the embodiment where test results are used for selection of parameters on a patient specific basis. Greater TWR reflects better performance and lower RRMSE reflects better performance.

N-back performance: N-back performance was measured by determining the percentage of correct letters recalled during a 30 second block and the total number of errors committed during a block (Voelcker-Rehage et al., 2006).

Dual-task Analysis: To examine participants' performance under the dual-task conditions, the dual task loss (DTL) was computed using a standard measure to compare performance on single and dual-task conditions (Lindenberger, U. et al., “Memorizing while walking: increase in dual-task costs from young adulthood to old age,” Psychol. Aging 15, 417-436 (2000), the entire contents of which is hereby incorporated by reference herein). The DTLs were computed as the percentage of loss in motor and cognitive performance during dual-task conditions relative to performance in the single-task conditions in the following manner:

DTL_{force}=[(mean dual-task_{force}−mean baseline/mean baseline_{force}]×100.

DTL_{n-back}=[(mean dual-task_{n-back}−mean baseline_{n-back})/mean baseline_{n-back}]×100.

This is a measure that essentially determines the cost from a motor and cognitive perspective of moving from a single task to the more complex and difficult dual-task.

### Statistical Analysis

Motor (RRMSE, TWR) and cognitive (percentage of correctly repeated letters (PRL), number of errors (NE)) performance data were analyzed with repeated measures ANOVAs (analysis of variance). Greenhouse Geyser adjustment was reported when the sphericity assumption was violated. Post-hoc contrasts (Bonferroni adjustment) were used to determine differences between the DBS status and level of task difficulty to determine the conditions that were most affected by the different DBS parameter settings. Analyses were conducted separately for the motor and cognitive task. These statistical methods can be applied according to the embodiment in which parameters are selected based on the test results.

Two 3 (DBS condition: Off DBS, Clinical DBS, Model DBS)×3 (task difficulty: O-back, 1-back, 2-back)×2 (context: single-task, dual-task) repeated measure ANOVAs were used to determine differences between different DBS parameter settings in n-back difficulty and between single- and dual-task context using PRL and NE. The repeated measure ANOVAs can be used when the study design is a within subject repeated measure, such that multiple measures on the same patient are obtained, but under varying conditions. Additionally, two 3 (DBS condition)×4 (task difficulty: force only, force at 0-back, 1-back, and 2-back difficulty) repeated measure ANOVAs were carried out using the RRMSE and TWR scores.

To examine whether DTLs for the force maintenance task and the n-back difficulties were significantly different from zero, a series of one-sample t tests (test value=0) were conducted separately for each DBS condition. Repeated measures ANOVAS with corresponding post-hoc tests were used to compare the DTLs for task difficulties (O-back, 1-back, 2-back) and DBS status. If there is no cost in moving from a single to a dual task, then the DTL would be zero.

### Results

### Clinical Ratings

Table 1 contains UPDRS-III Motor scores for each patient during Off, Clinical, and Model DBS. For all patients, the UPDRS-III scores decreased (and lower is better) with Clinical and Model DBS compared to Off DBS. Clinical DBS, on average, resulted in a 46 percent improvement in UPDRS-III ratings (range: 37 to 58 percent) while Model DBS also improved clinical UPDRS-III ratings by 46 percent (range: 38 to 59 percent). Statistical analysis (t-tests for paired samples) revealed that UPDRS-III scores for Clinical and Model DBS were significantly better than Off DBS (tcli-off(9)=3.90, p=0.004; tmod-off(9)=3.30, p=0.009). However, there was no statistical difference in UPDRS-III scores between Clinical and Model DBS settings (t(9)=0.23, p=0.820).

### DBS Power Consumption

The power consumption associated with Clinical and Model parameters for each stimulator and the total amount of power, in microwatts, is provided in Table 4. In terms of total power consumption, the Model parameters consume approximately 50 percent less microwatts than Clinical parameters (t_{mod-cli}(9)=8.45, p<0.0001). For all 10 patients, total power consumption was less with Model compared to Clinical parameters and power consumption was less with Model compared to Clinical parameters for both the right and left stimulators.

TABLE 4
Power consumption, in microwatts, for Clinical and Model
stimulation parameters for each side and total power
requirements for Clinical and Model parameters.
Power (μW)

| | Clinical | | Model | | Total | |
|---|---|---|---|---|---|---|
| Patient | Left | Right | Left | Right | Clinical | Model |
| 1 | 122.65 | 191.63 | 25.67 | 31.69 | 314.28 | 57.36 |
| 2 | 174.53 | 146.13 | 53.56 | 38.35 | 320.66 | 91.91 |
| 3 | 100.79 | 100.79 | 41.91 | 53.56 | 201.58 | 95.47 |
| 4 | 106.63 | 89.60 | 25.67 | 62.12 | 196.24 | 87.79 |
| 5 | 161.19 | 189.18 | 53.56 | 62.12 | 350.37 | 115.68 |
| 6 | 71.31 | 81.13 | 45.64 | 53.56 | 152.44 | 99.20 |
| 7 | 220.89 | 220.89 | 49.52 | 17.83 | 441.79 | 67.35 |
| 8 | 127.36 | 84.25 | 25.67 | 45.64 | 211.62 | 71.31 |
| 9 | 66.63 | 66.63 | 25.67 | 45.64 | 133.27 | 71.31 |
| 10 | 115.46 | 115.46 | 45.64 | 31.69 | 230.92 | 77.33 |
| Mean | 126.75 | 128.57 | 39.25 | 44.22 | 255.32 | 83.47 |
| SD | 47.43 | 54.77 | 12.21 | 14.39 | 97.70 | 17.62 |

### Cognitive Functioning and DBS During Single and Dual-Task Conditions

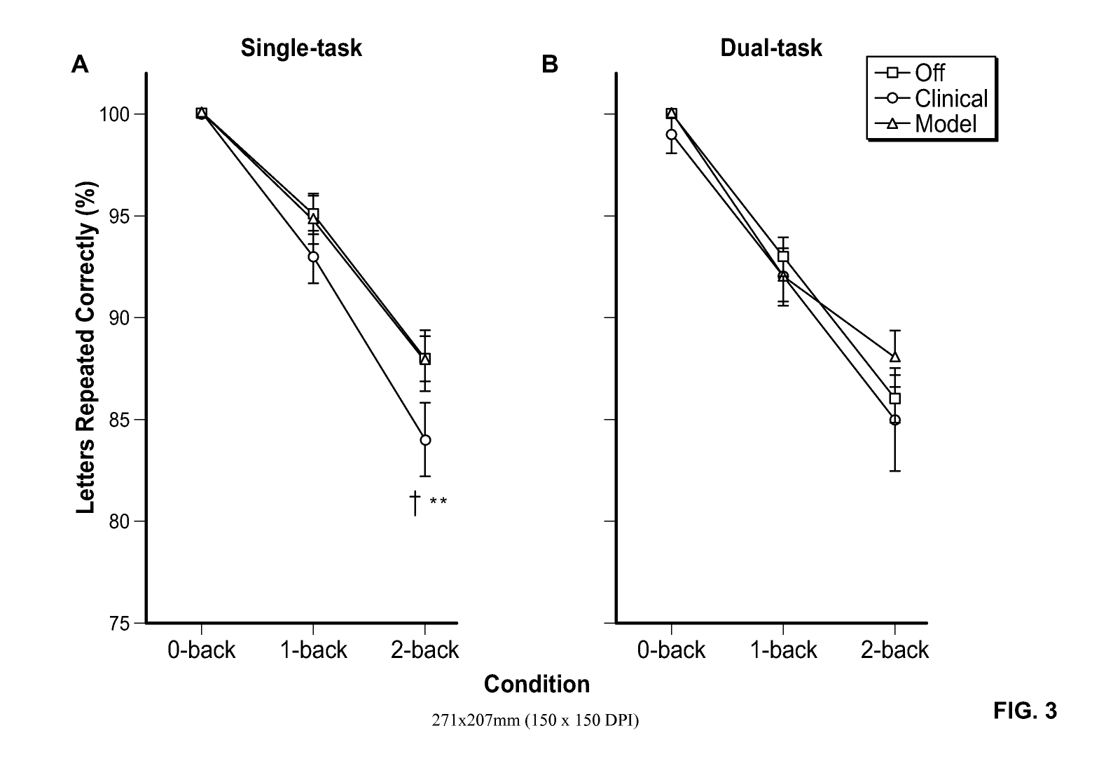

Percentage of Correct Letters (PCL): The results from the repeated measures ANOVA (cf. FIG. 3) revealed that overall n-back performance decreased with increasing task difficulty (F(2, 18)=48.422, p<0.001, η²=0.843). The main effects of DBS status (F(2, 18)=2.010, p=0.163) did not achieve statistical significance while the main effect of context (F(2, 18)=4.879, p=0.055) approached statistical significance. The task difficulty×DBS condition interaction, however, was significant (F(4,18)=2.945, p=0.033, η²=0.247), resulting from a greater performance decrease with increasing n-back difficulty for Clinical DBS than for Off and Model DBS. Performance on the 2-back during Clinical DBS was significantly lower than performance at Off DBS or Model DBS in single-task conditions. As task difficulty increases as a result of an increase in cognitive demands of the dual-task performance, declines would be found during Clinical DBS, but not during Model DBS.

Number of Errors (NE): Errors in cognitive function were primarily due to responding with the incorrect letter and the participant reporting to experimenter that they did not remember the letter to be recalled. Less than 0.5 percent of the errors were the result of the patient not responding within the ˜1.5 second time period. For the number of errors, the effect of task difficulty (F(2, 18)=50.381, p<0.001, η²=0.848) and the task difficulty by context interaction (F(2, 18)=3.859, p=0.040, η²=0.300) were significant. Participants produced more errors as the difficulty of the n-back task increased. The number of errors, however, did not significantly differ between the DBS states (F(2, 18)=0.450, p=0.644). This can occur, for example, as a function of the number of letters presented. For example someone can perseverate on a response and not get as many letters presented to that person.

### Motor Function and DBS During Single and Dual-Task Conditions

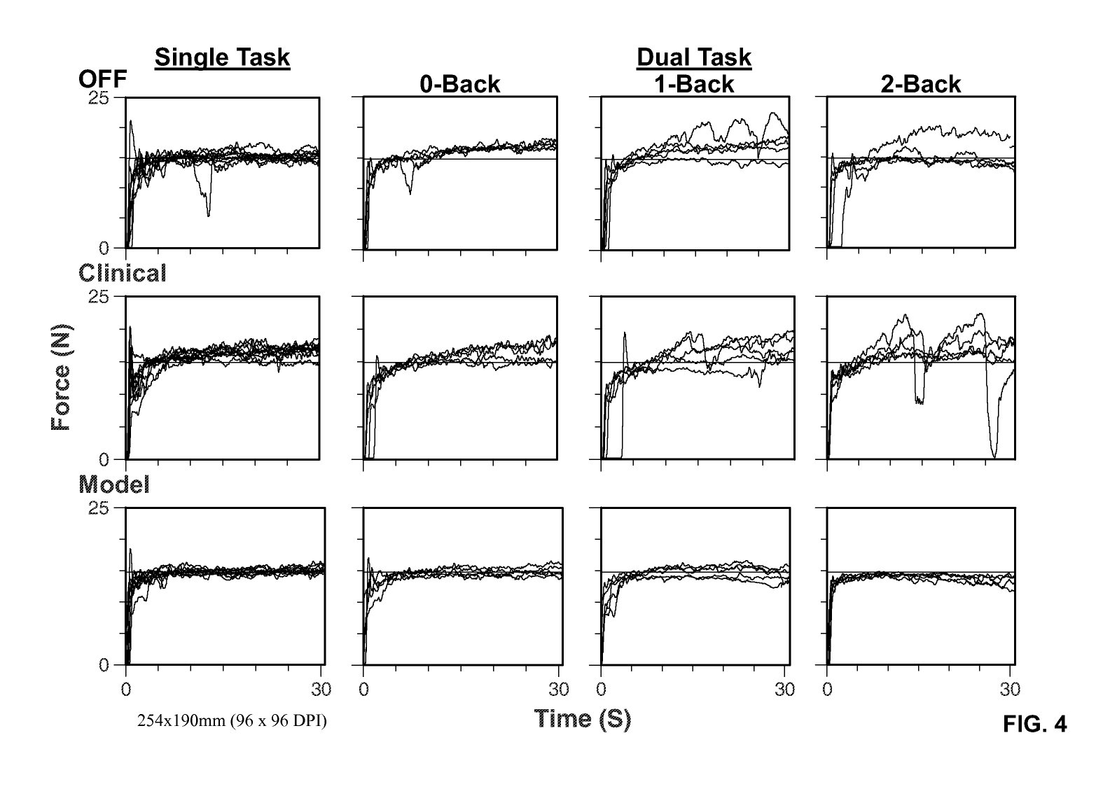

Representative force-tracking data for an entire set from one patient for all three DBS conditions during single and dual-task settings are presented in FIG. 4. When performing the force-tracking task only (left plots), Clinical and Model DBS resulted in better tracking performance compared to Off DBS.

While patients were Off, force tracking performance became slightly more variable as the difficulty of the dual-task increased. During Clinical DBS, middle plots, force-tracking performance declined dramatically as task difficulty increased, in particular during the 2-back condition in which variability was greatest. The lower panels depict force-tracking trials during Model DBS. In general, the consistency of force tracking was relatively unaffected by increasing task difficulty under dual-task conditions. The TWR and RRMSE measures were used to quantify force-tracking performance.

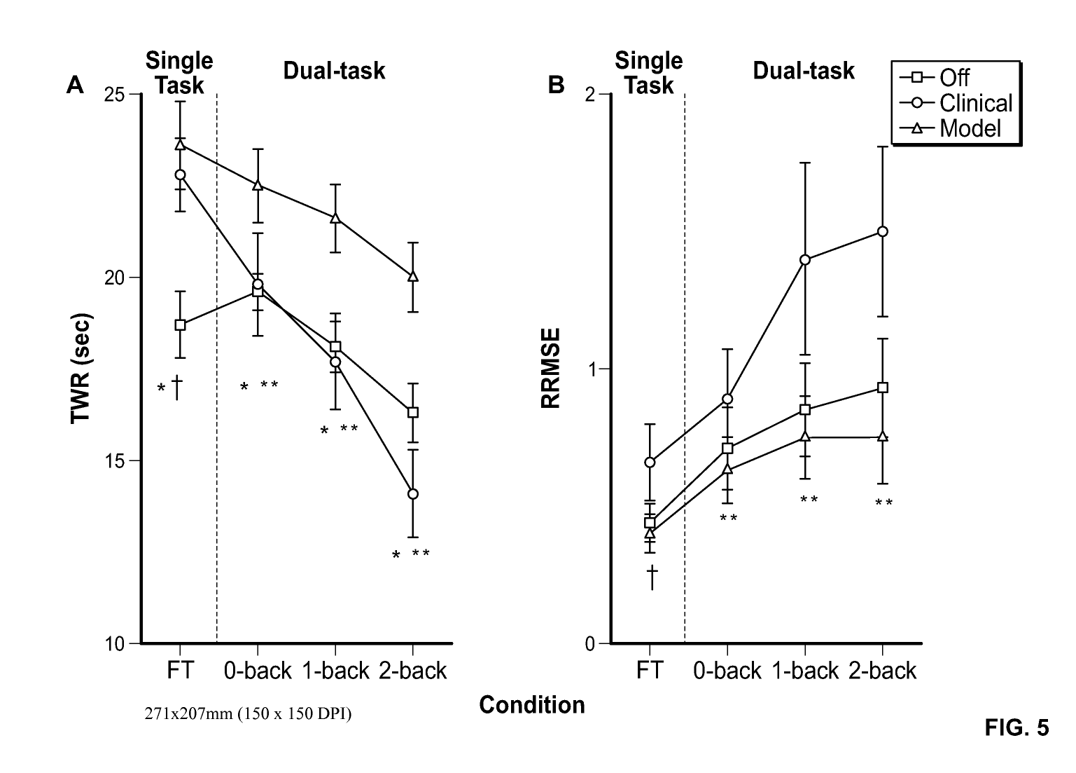

Time within Target Range (TWR): When completing the force maintenance task only, Clinical and Model DBS conditions were significantly better than the Off DBS condition. As expected, motor performance tended to decrease (lower TWR) as patients moved from the single to dual-task conditions (cf FIG. 5a). However, the rate of decline in motor performance differed across stimulation conditions. With Clinical DBS the rate of motor performance decline was greater compared to the decline under Model DBS settings. A significant interaction between DBS condition and task difficulty was present (F(6,54)=4.857, p<0.001, η²=0.351). During Off and Model DBS conditions, the slope of decline in motor performance was similar across dual-task conditions. However, under Clinical DBS settings, TWR decreased dramatically across all task difficulties. Furthermore, Model DBS led to significantly better force tracking performance as compared to Clinical DBS or Off DBS in all dual-task conditions.

Relative root mean square error (RRMSE): In general, the variability in force tracking increased significantly as task difficulty increased, moving from single to dual-task conditions (F(1.35, 27.73)=10.113, p=0.005, η²=0.529). Additionally, the force variability differed between the three DBS conditions (F(2,54)=5.042, p=0.018, η²=0.359), and the greatest variability occurred under Clinical DBS. In the dual-task conditions, Clinical DBS resulted in significantly worse performance than Off and Model DBS (cf. FIG. 5b). As shown in FIG. 5b, Clinical DBS resulted in more variable force production across conditions; as task difficulty increased to the 2-back condition, force variability was significantly greater compared to Model DBS.

Dual-task losses (DTLs) different from zero: The DTLs for n-back performance at the 0-back condition were relatively small and non-significant across the three DBS testing conditions. Declines in n-back performance were greater when moving from the single task 1-back condition to the dual-task 1-back condition, in particular for the Off DBS and Model DBS conditions (due to the fact that under single task conditions n-back performance was relatively high). In study data, the DTLs associated with Clinical DBS were not significantly different from the DTLs associated with Model DBS. From a cognitive perspective, the cost in performance when moving from single- to dual-task conditions was not statistically significant for any of the stimulation conditions. A reason for this may be that, despite the fact that patients reported attending to both tasks equally, they may have placed greater emphasis or allocated more attentional resources to performing the working memory task compared to force-tracking.

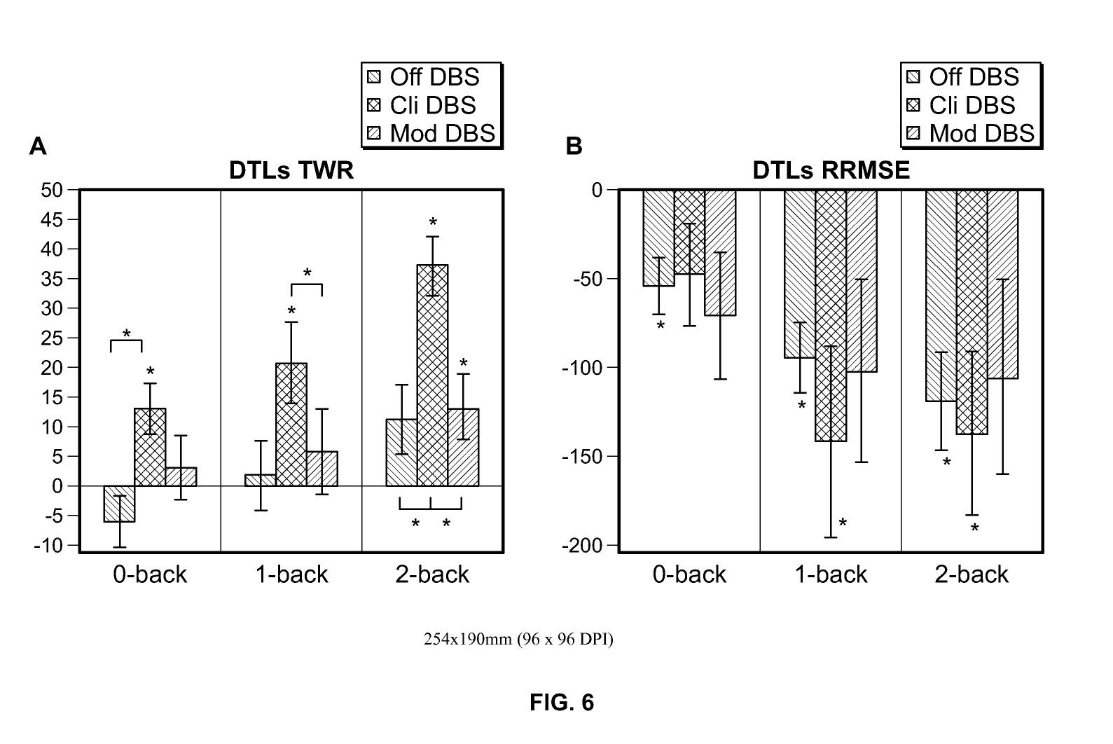

As expected, force tracking performance did decline as task complexity increased from single to dual-task conditions while Off DBS and under Clinical and Model DBS settings. However, the declines in force tracking, FIGS. 6a and 6b, were most present during Clinical DBS settings. For TWR, the greatest declines in motor performance when moving from a single to dual-task were associated with Clinical DBS (Clinical DBS: t_{0-back}(9)=3.091, p=0.013; t_{1-back}(9)=3.058, p=0.014; t_{2-back}(9)=7.151, p<0.001; Model DBS: t_{0-back}(9)=0.537, p=0.604; t_{1-back}(9)=0.771, p=0.460; =t_{2-back}(9)=2.363, p=0.042; Off DBS: t_{0-back}(9)=−1.542, p=0.157; t_{1-back}(9)=0.269, p=0.794; t_{2-back}(9)=2.026, p=0.073). The greatest performance decrements for each DBS condition occurred during the most complex testing condition, 2-back+force maintenance (compared to just force maintenance without the n-back test), and the smallest decrement during the simplest, 0-back+force maintenance (cf. FIG. 6a) (compared to just force maintenance without the n-back test). That is, as complexity of the task is increased, the quality of performance decreases, A similar pattern of results was present when examining the variability of force production (RRMSE): t_{0-back}(7) 3.54, p=0.01; t_{1-back}(7)=3.33, p=0.01; t_{2-back}(7)=7.42, p<0.01) (cf. FIG. 5b). The greatest declines in motor performance were associated with Clinical DBS (t_{0-back}(9)=−1.674, p=0.128; t_{1-back}(9)=−2.636, p=0.027; t_{2-back}(9)=−2.970, p=0.016). The DTLs in force tracking performance (RRMSE) at Off DBS were significant for all n-back conditions (t_{0-back}(9)=−3.767, p=0.004; t_{1-back}(9)=−5.023, p=0.001; t_{2-back}(9)=−4.131, p=0.003), whereas under Model DBS DTLs were not significant (t_{0-back}(9)=−2.014, p=0.075; t_{1-back}(9)=−2.005, p=0.076; t_{2-back}(9) (9)=−1.924, p=0.087).

Task difficulty and Stimulation differences in DTLs: The DTLs_{n-back}, in general, increased significantly as task difficulty also increased, (F(2, 18)=3.831, p=0.041, η²=0.299). However, the DTLs_{n-back} were not differentially affected across stimulation conditions (Off, Clinical or Model) (F(2, 18)=0.425, p=0.660).

For the DTLs_{force}, a significant main effect of task difficulty for TWR was present (F(2, 18)=26.984, p<0.001, η²=0.750). As task difficulty increased, DTLs in force maintenance also increased as shown in FIG. 6a. The loss in motor performance was relatively small for the 0-back condition while relatively large for the 2-back dual-task condition. A significant main effect of stimulation (F(2, 18)=5.940, p=0.010, η²=0.398) was present. Differences between DBS states were significant in the 0-back, 1-back and 2-back conditions (significantly higher DTLs with Clinical DBS compared to Off and Model DBS). The DTLs in terms of the variability (RRMSE) of force production were similar to TWR as losses in performance were greater during Clinical compared to Off and Model DBS conditions (FIG. 6b).

Recently, it has been shown that bilateral STN DBS disrupts PD patients' cognitive-motor functioning under dual-task conditions (Alberts et al., 2008). These DBS related declines in cognitive-motor functioning are minimized through the use of patient-specific DBS models that account for electrode location and the VTA. In an example embodiment, the primary criterion for the selection of DBS parameters can be maximized stimulation coverage of a target volume that includes the dorsal STN and white matter dorsal to the STN, thus minimizing stimulation of non-motor regions of the STN.

The typical clinical method of DBS programming was compared, with respect to cognitive-motor performance in advanced PD patients, to the computational approach described herein for selecting DBS parameters that minimize stimulation of non-motor regions of the STN. Clinical assessments indicated both methods of DBS programming were effective in improving UPDRS-III scores. However, under all dual-task conditions motor performance was, in general, better with Model determined stimulation parameters compared to Clinical settings. In addition, cognitive performance (working memory) was better during modestly complex task conditions, using Model compared to Clinical settings. Overall, these data suggest that cognitive-motor declines associated with bilateral STN DBS can be mitigated through the use of software that depicts the VTA associated with a given parameter setting relative to the targeted brain structure.

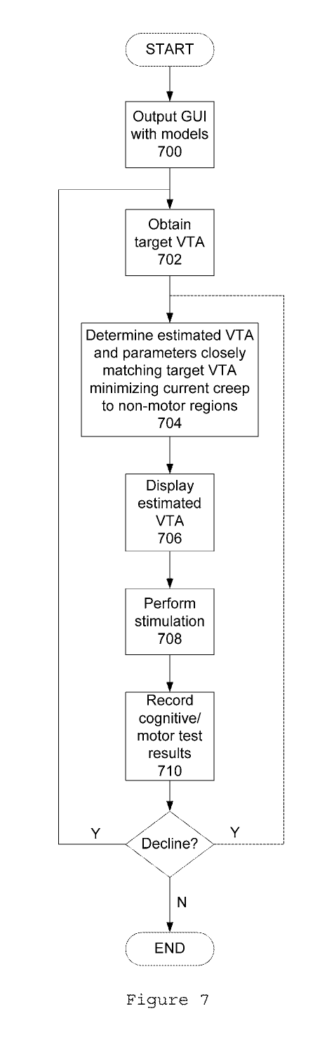

Referring to FIG. 7, in an example embodiment of the present invention, a system can, at step 700, output a GUI including a display of a model of a patient anatomy, e.g., the patient's brain, co-registered with a model of a stimulation leadwire. The brain model can be generated, for example, by fitting a brain atlas to images of the patient's brain. Alternatively, the images themselves can be displayed. Alternatively, the system can display the images and the model co-registered with each other.

At step 702, the system can obtain user input identifying a target VTA. The target VTA can be drawn such that it does not include more than 10% of the non-motor region of the patient brain, and specifically less than 10% of globus pallidus. In an example, the target VTA can be drawn such that it does not include any of the non-motor region.

At step 704, the systems and methods can determine an estimated VTA and corresponding stimulation parameters whose stimulation is estimated to produce the estimated VTA, which estimated VTA most closely matches the obtained target VTA. In an example embodiment, the estimated VTAs (and corresponding stimulation parameters) from which the system can select can be limited to those that do not extend outward beyond any of the perimeter of the target VTA, such that if the closest estimated VTA extends beyond the target VTA, a VTA that is less of a match but is completely included within the area of the target VTA would be selected. The estimated VTAs can be calculated based on predetermined functions and/or based on a patient population as further described in the '330, '312, '340, '343, and '314 applications.

In an alternative example embodiment, the system can be initially configured with a universal target VTA drawn to the generic model which is then mapped to the specific patient, separate input of a target VTA for each patient not being necessary. The system can provide a patient-specific closest matching estimated VTA and associated stimulation parameters based on the universal target VTA as applied to the patient model and based on a currently used electrode leadwire.

The clinician can use the output parameters for bilateral DBS stimulation for the patient. Because the parameters correspond to an estimated VTA that closely matches the target VTA which does not include stimulation of non-motor regions, or at least only up to 10% of such regions of the brain, and specifically less than 10% of globus pallidus, there would be significant improvement with respect to cognitive and/or motor-cognitive degeneration as compared to conventional bilateral DBS stimulation.

At step 706, the systems and methods can display the estimated VTA overlaid on the patient brain/leadwire model. For example the system can remove the target VTA from display, the estimated VTA being displayed in its place.

In an example embodiment of the present invention, cognitive, motor, and cognitive-motor function of the patient can be assessed to fine tune the stimulation parameters. For example, at step 708, the stimulation parameters corresponding to the estimated VTA can be used in a stimulation of the patient brain. Instead of the stimulation parameters corresponding to the estimated VTA, the system can allow for the clinician to provide input to modify the stimulation parameters, e.g., directly or by shifting the displayed estimated VTA or a displayed current field.

While the patient undergoes such stimulation, motor and cognitive tests, e.g., the combination of the n-Back test and the force-maintenance task as described above, can be administered. The system, at step 710, can record results of such tests. For example, the system can record and/or calculate the data corresponding to the graphs shown in FIGS. 3-6. With respect to force-maintenance, the system can include a force sensor that senses the force exerted by the patient, and can record such figures and determine the difference of such sensed force to a target force. The system can also output audio through a speaker listing a series of letters and can receive speech input via a′ microphone repeating letters for the n-back test. The system can compare the speech input to recorded letters that had been output to determine the correctness of the speech input. Alternatively, a clinician can administer the tests, e.g., offline.

If the results show a decline in motor, cognitive, and/or motor-cognitive function, the clinician can input a new target VTA, so that the method returns to step 702. Otherwise, the method can end.

In an example embodiment, the system can be preconfigured with predefined metrics concerning results of the administered tests, indicating acceptable results and unacceptable results. For example, the system can be configured with such indications concerning TWR, RRMSE, and DTLs with respect to motor and/or cognitive skill as appropriate. In response to unacceptable results, the system can (as reflected by the broken lines) automatically cycle back to, for example, step 704 to determine a new set of parameters and associated estimated VTA which can improve such patient functions. For example, the system can select parameters that produce a VTA with less stimulation of non-motor regions of the brain or whose edges are further from such regions of the brain.

According to an example embodiment, the systems and methods can record and visually identify which explored VTAs were associated with a side effect. The clinician can identify a VTA for which there are subpar results of the described tests as such VTAs. Additionally or alternatively, the system can automatically record such VTAs as being associated with a side effect.

Such recordation can be helpful in that, for example, the system can output a GUI showing explored regions and indicate which of those have been associated side effects, so that the clinician has more information on which to base selection of stimulation parameters during subsequent stimulation sessions.

In an example embodiment of the present invention, after determining the stimulation parameter settings, e.g., based on automatic or manual selection of parameters corresponding to a VTA that is closest to a target VTA that avoids the non-motor regions of the brain, and or based on results of motor function, cognitive function, and dual motor-cognitive function tests, a voltage of an electrode can be decreased if a selected voltage is determined to cause a tingling sensation in the patient stimulated with the determined stimulation parameters.

While is has been reported that when memory demands of a task were increased, PD patients with bilateral STN DBS exhibited deficits in working memory (Hershey et al., 2004), it has been determined that unilateral STN DBS has little effect on working memory as n-back performance was similar during unilateral stimulation to that when patients were off DBS. (Alberts et al., 2008). In the current study, with respect to bilateral STN DBS, n-back performance at the most difficult condition (2-back) was compromised to a greater degree under Clinical DBS than under Model DBS or when Off DBS. These data suggest that minimizing current spread to the non-motor regions of the STN can alleviate some of the declines in working memory that can be associated with bilateral STN DBS. While the use of Model parameters did mitigate working memory declines, compared to Clinical parameters, working memory during bilateral STN DBS with Model parameters was not better than performance during unilateral STN DBS (Alberts et al., 2008). The observation that cognitive functioning (working memory) during unilateral DBS was better than bilateral STN DBS, whether Model or Clinical based, provides a rationale for taking a more conservative approach to the implantation of DBS systems.

Therefore, according to an example embodiment of the present invention, a stimulation method can include implementing a staged DBS implantation strategy, by initially performing unilateral DBS, assessing the impact of the unilateral DBS, e.g., on cognitive function, and subsequently implanting the second side. Such a method can decrease the likelihood of cognitive declines that can be associated with bilateral STN DBS and which can ultimately diminish the patient's overall quality of life. For example, the unilateral DBS can be determined to be sufficiently effective, and the bilateral DBS can be delayed for 6-12 months or even as long as 5 years, thereby delaying the increased cognitive impairment that is a result of the bilateral DBS.

### Second Study: Postural Stability

In a second study according to the present invention, STN DBS was used to assess postural stability under dual-task (working memory+postural stability) conditions using DBS parameters determined through Clinical DBS and Model DBS. Standardized clinical and biomechanical data were collected from one advanced PD patient under three stimulation conditions: Off, Clinical, and Model DBS. Model-based settings were selected to minimize the spread of current to non-motor regions of the STN, thereby focusing stimulation on areas previously shown to produce ideal therapeutic benefit.

DBS in the STN has provided significant acute and chronic improvements in rigidity, bradykinesia and tremor; however, it is well documented that bilateral STN DBS may only improve gait in PD patients similar to their best medication “on” state (Allert. N. et al., “Effects of bilateral pallidal or subthalamic stimulation on gait in advanced Parkinson's disease,” Movement Disorders 16(6), 1076-85 (2001), the entire contents of which is hereby incorporated by reference herein). Studies also showed a progressive decline in gait and postural instability scores that do not appear to be due to progression of the disease (Tagliati, M. et al., “Long-Term Gait Deterioration after Bilateral STN DBS is not due to the Natural Progression of Parkinson's Disease,” American Academy of Neurology, Chicago, Ill. (2008), the entire contents of which is hereby incorporated by reference herein). Given the failure of current therapies to adequately manage gait and balance disorders in patients with PD, a new surgical target has emerged for the treatment of PD postural instability, the peduculopontine nucleus (PPN). The effect of PPN on the motor symptoms of PD has not been studied in the same detail as that for STN and GPi and thus far there is only limited and, in some cases, controversial data concerning its beneficial effect on parkinsonian motor symptoms.

Although maintaining a steady posture has been described as an automatic or a reflex controlled task, it does require attentional resources (Woollacott, M. et al., “Attention and the control of posture and gait: a review of an emerging area research,” Gait Posture 16(1), 1-14 (2002), the entire contents of which is hereby incorporated by reference herein). The aim of this second study was to assess whether bilateral STN DBS could effectively improve postural stability using a neuro-computational modeling approach to DBS parameter selection that has been shown to decrease cognitive declines in PD patients. It was hypothesized that minimizing the spread of DBS current to the non-motor territories of the STN would free up cognitive resources that could then be allocated to maintaining a more steady posture.

The second study involved a 51-year-old male diagnosed with idiopathic Parkinson's disease at age 39 with right-sided tremor. He was considered to have lower extremity gait and postural stability dysfunction, had medically intractable tremor, and experienced the wearing off phenomenon. Nine years after diagnosis he underwent simultaneous bilateral STN-DBS. Despite improvements of the wearing off phenomenon and tremor, he continued to experience postural instability. His stimulation parameters were set through traditional clinical methods and were stable two years prior to study participation. Model based parameters were determined using Cicerone visualization software, targeting the motor area of each STN using methods described above with reference to the first study. Table 5 contains the clinical and model DBS parameters used in the second study.

TABLE 5
Clinical and Model-based stimulation settings

| | Clinical | | | | Model | | | |
|---|---|---|---|---|---|---|---|---|
| | | | Pulse | | | | Pulse | |
| | | Voltage | Width | Frequency | | Voltage | Width | Frequency |
| | Contact | (V) | (μs) | (Hz) | Contact | (V) | (μs) | (Hz) |
| Left | 1-C+ | 3.2 | 90 | 135 | 2-C+ | 2.4 | 60 | 130 |
| Right | 2-C+ | 3.2 | 60 | 135 | 2-C+ | 1.8 | 60 | 130 |

### Materials and Methods

Blinded UPDRS-III evaluations were performed under three stimulation conditions: Off, Clinical and Model. All conditions were completed while the patient was in the ‘practically defined off’ anti-parkinsonian medication. Details regarding the experimental procedures and the process of determining Model-based parameters are described above with reference to the first study.

### The N-Back Task

Working memory was assessed with the n-back task. The n-back task is similar to the n-back task described above with reference to the first study. The procedure required verbal information to be maintained and updated in working memory needed for complex cognitive tasks such as language comprehension, planning or reasoning. The randomized sequence of letters was presented while patient's responses were monitored. The n-back was administered with three levels of difficulty (0-, 1- and 2-back). Three 60-second trials were completed for each n-back level with 60-seconds of rest between tasks. Working memory performance was measured by calculating the percentage of letters responded correctly during each n-back trial.

### Postural Stability Task

The patient stood barefoot on a force platform (Kistler instrument AG, Winterthur, Switzerland) measuring 400 mm (width)×600 mm (length)×45 mm (depth) while wearing a safety harness. The patient was instructed to maintain an upright standing position with arms at his side while fixating on a static point on the wall approximately 3.8m away. Ground reaction forces were recorded at a frequency of 100 Hz during 60 seconds of quiet standing. The manufacturer's software (BioWare® Type 2812A 1-3, version 3.24) was used to calculate the three absolute components of the force. Quantitative analysis of the posturographic signal was performed by calculating the elliptical area of displacement, as well as the mean center of pressure (COP) velocity (average COP trial path length/time of the trial (60s)) in both the mediolateral (ML) and anteroposterior (AP) directions for each n-back condition. Mean velocity of COP was used as it has been shown to be the most reliable measure of postural sway (Brooke-Wavell, K. et al., “Influence of the visual environment on the postural stability in healthy older women,” Gerontology 48(5), 293-7 (2002), the entire contents of which is hereby incorporated by reference herein).

Traditionally, postural control is evaluated using COP movements with the assumption that an increased COP sway (COP area) is indicative of increased center of mass (COM) sway. However, increased COP area can indicate the adoption of an alternate postural control strategy (Panzer. V. P. et al., “Biomechanical assessment of quiet standing and changes associated with aging,” Arch. Phys. Med. Rehabil. 76(2), 151-7 (1995), the entire contents of which is hereby incorporated by reference herein). To capture these alternate postural control strategies, data analysis was performed using the best fit ellipse as previously described (Duarte. M. et al., “Effects of body lean and visual information on the equilibrium maintenance during stance,” Exp. Brain. Res. 146(1), 60-9 (2002), the entire contents of which is hereby incorporated by reference herein).

### Dual Task: N-Back and Postural Stability Simultaneously

The participant performed nine dual task blocks where he performed the n-back task and postural stability task simultaneously. The postural stability task was performed in a random combination with each of the three n-back conditions (0-, 1-, and 2-back), three repetitions each. Sixty seconds of rest was taken between trials. The patient was instructed to give equal importance to both tasks.

### Results

Both Clinical and Model DBS resulted in a significant improvement in UPDRS-III ratings, 25 and 30 percent respectively, compared to Off-DBS.

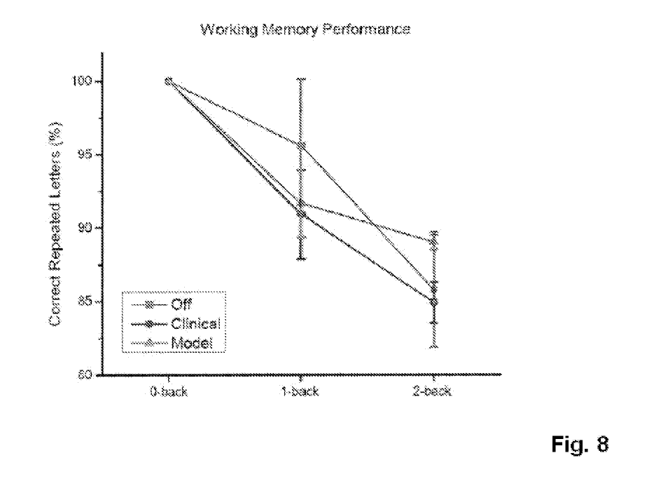

Overall, accuracy in the performance of the n-back task decreased with increasing task difficulty. FIG. 8 shows the percentage of letters repeated correctly under dual-task conditions. All stimulation conditions produced an equal percentage of correctly repeated letters under the O-back task. During the 1-back task, Off-DBS produced a greater percentage of correct repeated letters (96%) than both Model-based (92%) and Clinical DBS (91%). Under the 2-hack sequence for the dual task, Model-based DBS yielded the best performance with 89% of correct repeated letters as compared to 85% for Clinical DBS and 86% Off stimulation.

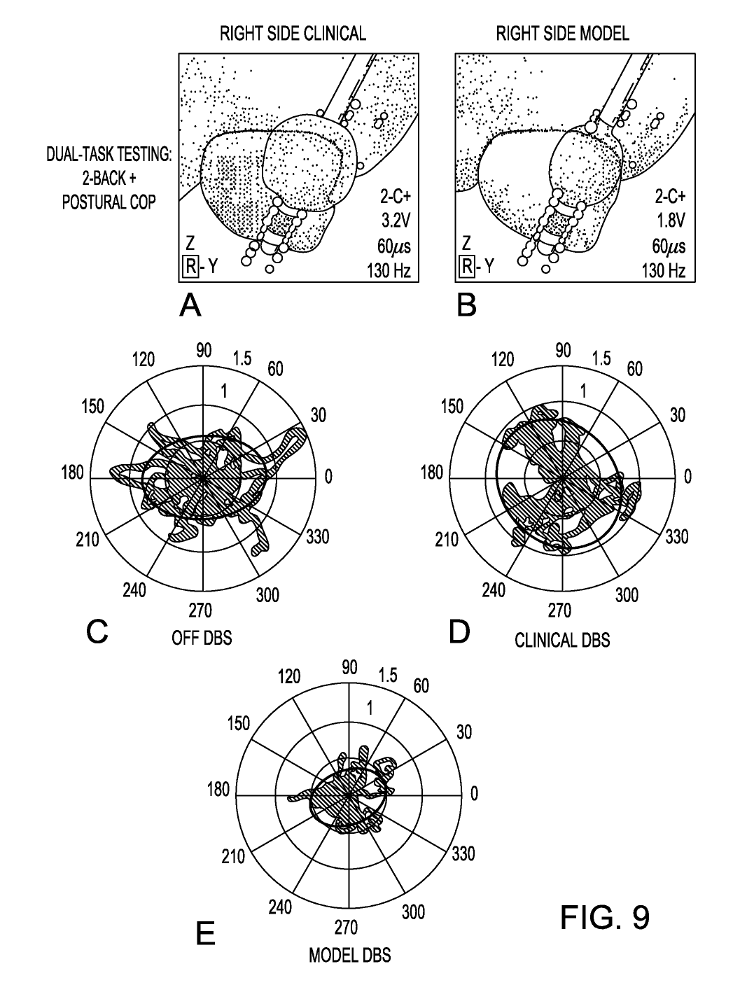

The patient's average COP area was the smallest when performing 2-back dual-tasks under Model-based DBS stimulation: 3.19 cm² compared to 5.29 cm² under Clinical DBS and 11.92 cm² under Off stimulation conditions. FIG. 9 shows the average AP and ML displacement as well as the elliptical area of displacement under the 2-back dual-task conditions.

Table 6 shows the mean COP velocities under each stimulation condition during all dual-tasks. Postural stability with Model-based DBS settings for AP sway resulted in a lower velocity value (2.6 mm/s) as compared to Clinical DBS settings (3.3 mm/s) and Off stimulation (4.3 mm/s). For ML sway, the velocity under Model-based settings (3.3 mm/s) was lower than Clinical DBS settings (4.1 mm/s) and Off stimulation (5.3 mm/s). Overall, Model-based settings produced smaller average velocity compared to Clinical DBS and Off stimulation, which suggests greater postural control.

TABLE 6
Summary of Variables for Average COP
Velocity Under Different Stimulator Settings

| | Sway | Velocity |
|---|---|---|
| Variable | direction | (mm/s) |
| Off Stimulation | AP | 4.3 |
| | ML | 5.3 |
| Clinical DBS Settings | AP | 3.3 |
| | ML | 4.1 |
| Model-based DBS Settings | AP | 2.6 |
| | ML | 3.3 |

Mean velocity is presented for antero-posterior (AP) and mediolateral (ML) sway direction for all variables.

In the second study, it was shown that DBS related declines were reduced under dual-task conditions with use of settings determined through visualization software that optimizes the volume of tissue activated in the STN region. The clinical assessments of the patient showed that both methods of programming, Clinical and Model-based DBS, were effective in improving motor scores assessed through UPDRS-III as compared to Off stimulation. Overall, cognitive performance using Model-based DBS was better during the complex-task conditions compared to Clinical DBS settings. The patient exhibited decreased postural sway as well as a decrease in the amount of cognitive errors under Model settings when compared to both Off stimulation and Clinical DBS settings.

Understanding how Parkinson's disease and DBS impact cognitive and postural performance under conditions requiring dual-tasks could result in a greater quality of life for PD patients. Activities in daily living generally require both cognitive and motor components. Because of the cognitive declines resulting from bilateral STN-DBS in advanced PD patients, routine activities can be a challenge. Bilateral STN-DBS side effects require the patient to place higher demands on cognition which results in less attention towards postural stability and locomotion. Therefore, by minimizing spread of current to the non-motor areas of the STN, and thus improving cognition, theoretically more attention could be placed on postural stability and gait function. In an attempt to treat the disabling postural deficits, surgeons have recently targeted the PPN. The current data would suggest that bilateral STN DBS has not fully been exploited for the treatment of postural instability of advanced PD patients. A computational modeling approach to improve STN-DBS programming can offer a solution for optimizing currently used techniques rather than stimulating a difficult target such as the PPN.

In the event of inconsistent usages between this document and those documents incorporated by reference herein, the usage in the incorporated reference(s) should be considered supplementary to that of this document; and for irreconcilable inconsistencies, the usage in this document controls.

The above description is intended to be illustrative, and not restrictive. Those skilled in the art can appreciate from the foregoing description that the present invention can be implemented in a variety of forms, and that the various embodiments can be implemented alone or in combination. Therefore, while the embodiments of the present invention have been described in connection with particular examples thereof, the true scope of the embodiments and/or methods of the present invention should not be so limited since other modifications will become apparent to the skilled practitioner upon a study of the drawings, specification, and following claims. For example, while example embodiments discussed in detail refer to PD patients, embodiments of the present invention, for example, pertaining to selection of stimulation parameters based on monitoring of cognitive function, motor function, and combination thereof, can be applied to patients having other neuro-degenerative diseases, including neuro-motor diseases or neuro-cognitive diseases.

What is claimed is:

1. A computer-implemented method, comprising:
selecting, by a computer processor, stimulation parameters for stimulating an anatomical region of a patient, the selection being based on results of at least one dual task test on which basis postural stability is assessed.

2. The method of claim 1, wherein the stimulation parameters are selected for treatment of a neuro-degenerative disorder, and the at least one dual task test is conducted on the patient to be stimulated using the selected stimulation parameters.

3. The method of claim 2, wherein the stimulation is performed using implanted electrodes of a deep brain stimulation (DBS) device.

4. The method of claim 3, further comprising:
calculating, in conjunction with the at least one dual task test, a computational model of estimated activated tissue for previously selected stimulation parameters to optimize the selecting of the stimulation parameters.

5. The method of claim 4, wherein the computational model provides a visualization of the volume of activated tissue relative to a patient-specific anatomical image and the implanted electrodes.

6. The method of claim 2, wherein the motor function task includes the patient standing upright on a force platform while fixating at a distant target.

7. The method of claim 6, further comprising:
calculating displacement and center of pressure data as results of the motor function task; and
evaluating the data via a best fit ellipse to determine the patient's postural sway.

8. The method of claim 2, wherein the cognitive function task is an n-Back task used for evaluating working memory, the motor function task is used for evaluating postural stability, and wherein the patient is subjected to both the n-Back task and the postural stability task simultaneously.

9. The method of claim 1, wherein the selecting is performed to maximize stimulation of a target motor anatomical region and minimize current creep to non-motor anatomical regions.

10. The method of claim 9, wherein the anatomical regions are part of the subthalamic nucleus (STN).

11. The method of claim 1, wherein the stimulation parameters are selected to minimize stimulation-related cognitive declines.

12. The method of claim 1, wherein the dual task test includes performance of a motor function task used to evaluate postural sway.

13. The method of claim 1, wherein the parameters are selected to improve postural stability.

14. The method of claim 1, wherein the parameters are selected to improve cognitive function.

15. The method of claim 1, wherein the parameters are selected to improve both cognitive function and postural stability.

16. The method of claim 1, wherein the at least one dual task test includes a plurality of dual task tests.

17. The method of claim 1, wherein the dual task test tests cognitive and motor function, the motor functions including postural stability.

18. A computer-implemented method, comprising:
assessing, by a computer processor, a previously performed stimulation of an anatomical region of a patient by analyzing results of at least one dual task test, the results including an indication of postural stability.

19. A computer-readable medium having stored thereon instructions executable by a processor, the instructions which, when executed by the processor, cause the processor to perform a method, the method comprising:
selecting stimulation parameters for stimulating an anatomical region of a patient, the selection being based on results of at least one dual task test on which basis postural stability is assessed.

20. A system, comprising:
a computer processor configured to select stimulation parameters for stimulating an anatomical region of a patient, the selection being based on results of at least one dual task test on which basis postural stability is assessed.
